(19) United States
(12) Patent Application Publication
(10) Pub. No.: US 2007/0106138 A1
(43) Pub. Date: May 10, 2007

(54) Intraoral apparatus for non-invasive blood and saliva monitoring & sensing

(76) Inventors: Ben Zion Beiski, Kiryat-Ono (IL); Andy Wolff, Harutzim (IL)

Correspondence Address: Martin D. Moynihan;PRTSI, Inc., P.O. Box 16446, Arlington, VA, 22215, US

(21) Appl. No.: 11/603,897

(22) Filed: Nov. 24, 2006

Related U.S. Application Data
(63) Continuation-in-part of application No. PCT/IL05/00542, filed on May 26, 2005.

(51) Int. Cl. A61B 5/05; A61B 5/00; A61B 5/02

(52) U.S. Cl. 600/349; 600/549; 600/573; 600/500; 600/365

(57) ABSTRACT

Controlled-specimen-sampling oral devices are described, implanted or inserted into an oral cavity, built onto a prosthetic tooth crown, a denture plate, braces, a dental implant, or the like. The devices are replaced as needed. The controlled specimen sampling may be passive, based on a dosage form, or electro-mechanically controlled, for a high-precision, intelligent, specimen sampling. Additionally, the controlled sampling may be any one of the following: sampling in accordance with a preprogrammed regimen, sampling at a controlled rate, delayed sampling, pulsatile sampling, chronotherapeutic sampling, closed-loop sampling, responsive to a sensor's input, sampling on demand from a personal extracorporeal system, sampling regimen specified by a personal extracorporeal system, sampling on demand from a monitoring center, via a personal extracorporeal system, and sampling regimen specified by a monitoring center, via a personal extracorporeal system. Specimen collection in the oral cavity may be assisted or induced by a transport mechanism, such as any one of, or a combination of iontophoresis, electroosmosis, electrophoresis, electroporation, sonophoresis, and ablation. The oral devices require replacement at relatively long intervals of weeks or months. The oral devices and methods for controlled specimen sampling apply to humans and animals.

### RELATED APPLICATIONS

This application is a continuation-in-part of PCT Patent Application No. PCT/IL2005/000542 filed May 26, 2005, which claims the benefit of U.S. Provisional Patent Application No. 60/574,562 filed May 27, 2004, the contents of which are incorporated herein by reference.

### FIELD OF THE INVENTION

The present invention relates to body-fluid sensing and monitoring, and more particularly, to oral devices, mounted on dental implements, configured for sampling and monitoring of blood, saliva, other oral fluids, such as sputum and crevicular fluid, and various other internal body fluids. Additionally, the present invention relates to methods of clinical sampling and analyzing, by the oral devices.

### BACKGROUND OF THE INVENTION

Dental Structure and Dental Implements:

The following is a brief overview of a tooth structure and of known techniques of dental repair and reconstruction, which relate to the present invention. FIG. 1 is a cross-sectional view of a tooth 10, as taught, for example, by www.dentalreview.com/tooth anatomy.htm. As seen in the figure, the basic parts of a tooth are: a crown 12, the portion of tooth above a gum 14, and a root or roots 16, which anchor the tooth in a jawbone 15. A pulp 18 is arranged within a pulp chamber 20 and within a root canal or root canals 22.

Crown 12 is formed of an inner structure of dentine 26 and an external layer of enamel 24, which defines a chewing surface 28. There may be one, two, or more roots 16. Each has an external layer of cement 30, inner structure of dentine 26, and one root canal 22. Pulp 18 is formed of tiny blood vessels, which carry nutrients to the tooth, and nerves, which give feeling to the tooth. These enter root canals 22 via accessory canals 32 and root-end openings 34.

Tooth 10 may define a cylindrical coordinate system of a longitudinal axis x, and a radius r. A coronal or incisal end 36 may be defined as the end above gum 14 and a apical end 38 may be defined as the end below it.

Various intraoral devices and dental reconstruction and repair methods that relate to the present invention are reviewed in conjunction with FIGS. 2A-7C, hereinbelow.

Bridge: A bridge may be used to fill a gap of up to four teeth, where there are healthy natural teeth on either side of the gap. FIGS. 3A-3F illustrate an application of a three-unit bridge 60 between two healthy teeth 62 and 64.

As seen in FIGS. 3A-3B, the dentist will prepare teeth 62 and 64 on either side of the gap by removing portions of the enamel and dentin, leaving stumps 66 and 68. Impressions or molds of stumps 66 and 68 and the gap between them are taken for the construction of the bridge. In the meantime, a temporary bridge is applied to protect the exposed stumps and provisionally restore the missing teeth.

As seen in FIGS. 3C-3D, the dentist then fits bridge 60, which includes a prosthetic tooth crown 70, over stumps 66 and 68. If the fit is good, he cements bridge 60 into place, restoring function to the area.

FIGS. 3E-3F illustrate an alternative technique: a bridge 72 may be formed of prosthetic tooth crowns 70 and anchors 74, adapted to clamp onto healthy teeth 62 and 64. Unlike bridge 60 of FIGS. 3C-3D, which is cemented into place, bridge 72 may be removed, for example, for cleaning.

Dental Implant: As an alternative to a bridge, a dental-implant-and-prosthetic-tooth-crown 80 may be used. As seen in FIGS. 4A-4C, dental-implant-and-prosthetic-tooth-crown 80 includes, for example, a dental implant or fixture 82, surgically implanted into the bone, which grows around it. Once dental implant 82 is anchored in the bone, a stump 84 is mounted on it and prepared to accept prosthetic tooth crown 70.

Dentures: When several teeth are missing, dentures 90 can be used, containing a plurality of prosthetic tooth crowns 70, as seen in FIGS. 5A-5C.

It is possible to get either full dentures, of all the teeth, as seen in FIG. 5A, or partial dentures, of fewer teeth, as seen in FIG. 5B. Full dentures are form-fitted to the gum ridges, creating an adhesive effect that keeps them in place. Partial dentures may be adapted to fit around the natural teeth, to help them stay in place. Additionally, as seen in FIG. 5C, a dental implant post 82 may be used to further to secure the dentures.

Crown: At times, the root of the tooth is intact. But its upper portion is severely decayed or broken. An artificial crown may then be placed on the tooth, as seen in FIGS. 6A-6C.

FIG. 6A illustrates a broken tooth 92. As seen in FIG. 6B, it is prepared by removing a portion of the enamel and dentin, exposing a stump 94. As seen in FIG. 6C, a crown 96 is then cemented over stump 94, restoring the chewing surface.

Braces: Other known dental devices include braces for orthodontics. FIG. 7A illustrates braces 100, which include molar bands 102, arch wires 104, and brackets 106.

Alternatively, FIG. 7B illustrates braces 110, which includes a metal or plastic plate 112, adapted to fit against the roof of the mouth, and wires 114 and 116. Alternatively, FIG. 7C illustrates invisible braces 120. In general, the braces of FIGS. 7A-7C may be easily removed, for example, for cleaning.

Transport Mechanisms

The following is a brief overview of transport mechanisms.

Electrotransport refers to at least one, and possibly a combination of transport mechanisms, which supplement the naturally occurring transfer modes.

Medical devices that include drug delivery by electrotransport are described, for example, in U.S. Pat. No. 5,674,196, to Donaldson, et al., U.S. Pat. No. 5,961,482, to Chien, et al., U.S. Pat. No. 5,983,131, to Weaver, et al., U.S. Pat. No. 5,983,134, to Ostrow, and U.S. Pat. No. 6,477,410, to Henley, et al., all of whose disclosures are incorporated herein by reference.

Electroosmosis involves the movement of a solvent with the agent through a membrane under the influence of an electric field.

Electrophoresis is based on migration of charged species in an electromagnetic field. Ions, molecules, and particles with charge carry current in solutions when an electromagnetic field is imposed. Movement of a charged species tends to be toward the electrode of opposite charge. The voltages for continuous electrophoresis are rather high (several hundred volts).

Electroporation is the process in which a biological barrier is subjected to a high voltage alternating-current (AC) surge, or pulse. The AC pulse creates temporary pores in the biological membrane, specifically between cells. The pores allow large molecules, such as proteins, DNA, RNA, and plasmids to pass through the biological barrier.

Iontophoresis is a method of transdermal local drug delivery using electrical current. A charged, ionic drug is placed on the skin with an electrode of the same charge, allowing direct current to drive the drug into the skin

Reverse iontophoresis is a process that uses electric current to draw biologically important species (such as glucose) from the sub-tissue space to the skin/tissue surface.

Sonophoresis is a transport method based on the use of ultrasonic means. By applying ultrasound vibrations (a low frequency of between 20 kHz and less than 1.5 MHz), rather than the therapeutic frequency, should be used) over the tissue, the movement through skin induces growth and oscillations of air pockets, a phenomenon known as cavitation. Sonophoresis has the advantage that the compounds do not have to be ionised. For example, in U.S. Pat. Nos. 6,002,961, 6,018,678, and 6,002,961 to Mitragotri, et al., U.S. Pat. Nos. 6,190,315 and 6,041,253 to Kost, et al. U.S. Pat. No. 5,947,921 to Johnson, et al. and U.S. Pat. Nos. 6,491,657, and 6,234,990 to Rowe, et al., all of whose disclosures are incorporated herein by reference.

Reverse Sonophoresis: The cavitation of the skin/tissue cells occurs as a result of applying sonophoresis over the tissue increases the permeability in both directions, allowing the extraction (and collection) of fluids, mainly of blood. The extracted fluids need not to be ionized.

Radio-frequency-driven micro-channeling: increasing the skin penetration for selected drugs using a microelectronic device based on ablation of outer layers of tissues (skin, mucosa) using radiofrequency high-voltage currents. These radiofrequency currents created an array of microchannels (across the stratum corneum deep into the epidermis in the skin).

Reverse Radio-frequency-driven micro-channeling—The cavitation of the skin/tissue cells, which occurs as a result of applying radio-frequency-driven micro-channeling over the tissue, increases the permeability in both directions, allowing the extraction (and collection) of fluids, mainly of blood. The extracted fluids need not to be ionized.

Membrane-coated collecting container consists of core coated by a substantially insoluble polymer, for example, polyvinyl chloride, wherein the coating is mixed with a water soluble, pore-forming compound.

Micro-needles a process for the fabrication of out-of-plane hollow microneedles in silicon. The fabrication method consists of a sequence of deep-reactive ion etching (DRIE), anisotropic wet etching and conformal thin film deposition, and allows needle shapes with different, lithography-defined tip curvature.

Reverse micro-needles array for extracting blood samples through the tissues.

Vacuum may be applied inside the capsule in synchronization to the activation of other permeability enhancer such as iontophoresis and/or sonophoresis, attracting the egress fluids into the reservoir.

Review of Art

U.S. Pat. No. 4,629,424 discloses a device for intraoral ambient sensing. In a specific embodiment, the device comprises a removable oral (Hawley) appliance containing a number of chemically sensitive electrodes and a common reference electrode at the chemical sensing sites and a telemetry unit plus power pack for signal transmission. This device may also be used to monitor non-chemical parameters, notably pressure, temperature, and flow. In this embodiment, the removable oral (Hawley) appliance contains a number of sensors to monitor the parameter of interest. For example, a pressure sensor configured as an artificial uvula and mounted to the oral (Hawley) appliance emulates actual uvula pressure events. To monitor chewing events, a pressure sensor is mounted to the oral (Hawley) appliance in such a manner that it is optimally located at an open bite location of the mouth. In either embodiment, a telemetry unit mounted to the oral (Hawley) appliance transmits the intraoral ambient information as radio frequency signal bursts. A remotely located receiver circuit receives the radio signals which are broadcast and recovers the information encoded in them.

U.S. Pat. No. 6,0010,463 describes a device and method for collecting a fluid sample and introducing it into a sensing device for real time analysis, thus providing flexibility and simplicity. Sample collection is accomplished with a disposable cartridge of the sensing device to permit quick and easy sample introduction and eliminate the risk of sample spillage. An exemplary embodiment of the collection device includes a capillary tube capable of receiving or drawing a fluid sample and introducing the fluid sample into the disposable cartridge, a reservoir chamber capable of receiving a fluid sample and a capillary tube holder capable of supporting one end of the capillary tube in the reservoir chamber to draw the fluid sample by capillary action. Alternative embodiments of the invention are used in combination with the variety of blood collection assemblies employed by the medical industry to collect fluid samples.

Other studies have used invasive instruments to measure the pH of the pharynx area to detect acid reflux. Instrument-based pH monitoring of the pharynx has shown results in a pH range of around 5 to 7.5 (Haase, G., et al., “A unique teletransmission system for extended four-channel esophageal pH monitoring in infants and children,” J. Ped. Surg., January 1987; 22(1): 68-74; Contencin, P., et al., “Measurement of pH of the rhinopharynx in children with gastroesophageal reflux,” Presse Medicale, 1989; 18(1): 13-6; Wiener et al, 1989, supra; and Chen et al, 1992, supra). Instrument-based pH testing during surgery determined that individual pharynx pH rarely varied by more than 1.0 pH unit in the absence of regurgitation (Joshi, G., et al., “Continuous hypopharyngeal pH measurement in spontaneously breathing anesthetized outpatients: laryngeal mask airway versus tracheal intubation,” Anesth. Analg., 1996; 82: 254-7). No previous studies have considered the gargling method for detecting acid reflux. Furthermore, no prior medical literature or patents have described the collection of fluid from gargling and measurement of the pH of this fluid, within a collection container or device as a means of evaluating for presence of acid reflux.

Several patents disclose methods involving the placement of devices in the oral cavity to obtain an oral sample for testing. These include, for example, U.S. Pat. No. 4,114,605, “Intraoral cup for collecting saliva and method of using the same” (McGhee et al.), U.S. Pat. No. 4,418,702, “Method and apparatus for collecting saliva” (Brown et al.), U.S. Pat. No. 5,103,386, “Oral collection device and kit for immunoassay” (Goldstein et al.), U.S. Pat. No. 5,339,829, “Oral collection device” (Thieme et al.), U.S. Pat. No. 5,479,937, “Oral collection device” (Thieme et al.), U.S. Pat. No. 5,563,073, “Personal blood alcohol level testing kit” (Titmas, T.) and U.S. Pat. No. 5,573,009, “Oral sample collection method” (Thieme et al.).

Another study (Ayre, J., “The gargle test: new oral cancer screening method,” N.Y. State Dent. J., June-July 1972; 38(6): 345-50) discusses collecting the fluid from gargling for subsequent laboratory analysis of collected tissue cells for evidence of malignancy.

Other literature describes gargling to obtain a sample of fluid for subsequent laboratory identification of the types and quantities of microbial organisms present in the pharynx.

U.S. Pat. No. 4,321,251, “Detection of malignant lesions of the oral cavity utilizing toluidine blue rinse,” (Mashberg, March, 1982) describes a method of rinsing and gargling with a specified solution to detect a color change within the mouth, for detection of malignant oral lesions.

U.S. Pat. No. 4,397,944, “Compositions for diagnosis of dental caries activity,” (Komura, 8/83) describes a method of detecting the pH of dental plaque placed in a specified solution, using bromothymol blue or other coloring agents.

U.S. Pat. No. 5,022,409, “Oral rinse immunoglobulin collection kit for immunoassay and method thereof” (Goldstein et al.) discloses a method of rinsing the mouth to collect an oral sample for subsequent storage and transport for testing.

U.S. Pat. No. 6,143,948, to Leitao, et al., “Device for incorporation and release of biologically active agents,” describes an implantable device coated with a layer of calcium phosphate and optionally one or more biologically active substances such as growth factors, lipids, (lipo)polysaccharides, hormones, proteins, antibiotics or cytostatics. The implant may be used for biomedical use, i.e. as a bone substitute, a joint prosthesis, a dental implant (prosthodontics), a maxillofacial implant, and the like, and as a point-of-care test performed by the health professional to provide a rapid result in a clinical setting. The methods of the invention may be applied to any characteristic of the mouth and/or pharynx which can be sampled by rinsing and/or gargling, retrieving the resulting fluid in a specified collection container or device and then analyzing the fluid in the collection container or device to provide a rapid or point-of-care result. More particularly, the invention provides methods and a test kit for non-invasive, non-instrumented assessment and diagnosis of the condition of acid reflux by sampling of the pharynx through the gargling process, and rapid determination of the pH of the collected fluid.

The following US patents have some relevance to the present invention: U.S. Pat. Nos. 4,629,424, 4,948,587, 5,458,140, 5,786,227, 6,010,463, 6,152,887 and 6,652,141.

The following publications have some relevance to the present invention:

Blue Cross & Blue Shield Association. Iontophoresis for medical indication. Technology Evaluation center 2003, Blue Cross & Blue Shield Association.

Boris Stoeber and Dorian Liepmann. DESIGN, FABRICATION AND TESTING OF A MEMS SYRINGE. Berkeley Sensor and Actuator Center, University of California at Berkeley Berkeley, Ca 94720-1774.

Chen M et al. Gastroesphageal reflux disease: correlation of esophageal pH testing and radiographic findings, Radiology, 1992; 185: 483-6.

Gardeniers H J G E, Luttge R, Berenschot E J W, de Boer M J, Yeshurun S Y, Hefetz M, van't Oever R, van den Berg A. Silicon micromachined hollow microneedles for transdermal liquid transport. J IEEE Microelectromech Syst 2003; 12: 855-862.

Hunter I et el. Minimally invasive glucose sensor and insulin delivery system, MIT Home Automation and Healthcare Consortium, Phase 2, final report, Sep. 30, 2000.

Merino V, Kalia Y N, Guy R H. Transdermal therapy and diagnosis by iontophoresis, Tibtech 1997, 15:288-90.

Merino V, López A, Guy R H. Noninvasive sampling of phenylalanine by reverse iontophoresis, Proceed Int'l Symp Control Rel Bioact Mater 25 (1998) Controlled Release Society Inc.

Mitragotri S, Farrell J, Tang H, Terahara T, Kost J, Langer R. Determination of threshold energy dose for ultrasound-induced transdermal drug transport, J Invest Dermatol 2003; 121: 1138.

Mitragotri S, Coleman M, Kost J, Langer R. Analysis of ultrasonically extracted interstitial fluid as a predictor of blood glucose levels 2000; 89:961-966.

Moon, Sang-Jun. Fabrication of microneedle array using inclined LIGA process Pohang University of Science and Technology Department of Mechanical Engineering, http://www.samsung.com/AboutSAMSUNG/SocialCommitment/HumantechThesis/down loads/9th/g1.pdf

Sekkat N, Naik A, Kalia Y N, Glikfeld P, Guy R H. Reverse iontophoretic monitoring in premature neonates: feasibility and potential, J Control Release 2002, 81:83-89.

Singh S et al. Determinants of esophageal ‘alkaline’ pH environment in controls and patients with gastroesophageal reflux disease. Gut 1993 34: 309-16.

Sintova A C, Krymberka I, Daniel D, Hannan T, Sohn Z, Levin G. Radiofrequency-driven skin microchanneling as a new way for electrically assisted transdermal delivery of hydrophilic drugs, J Control Release 2003, 89:311-320.

Terahara T, Mitragotri S, Kost J, Langer R. Dependence of low-frequency sonophoresis on ultrasound parameters; distance of the horn, intensity, and frequency, Int. Journal Pharm 235: 35-42.

### BRIEF SUMMARY OF THE INVENTION

The present invention relates to body-fluid sensing and monitoring, and to oral devices, mounted on dental implements, configured for sampling and monitoring of blood, saliva, other oral fluids, such as sputum and crevicular fluid, and various other internal body fluids. Additionally, the present invention relates to methods of clinical sampling and analyzing, by the oral devices.

More specifically, the present invention relates to devices and methods for non-invasive (or minimally invasive) blood and oral fluids sampling in a subject, by the collection of blood and oral fluids from the oral tissues and cavity of the subject. Blood and oral fluids are obtained from the tissues using mainly reverse drug delivery techniques such as iontophoresis, sonophoresis, RF microchanneling techniques. The sample is taken out to a sampling analyzer and/or analyzed by built-in sensors and the results are transmitted to an extraoral monitoring device or to a controlled drug delivery system or other therapeutic system, such as an electrostimulator to deliver the appropriate dosage or stimulation based on the sensing results.

Built-in analysis of the characteristic of interest in the fluid is performed in the specified collection device, providing instantaneous results without the manual handling of the fluid sample. The results are transmitted to a monitoring and logging device and/or used to close the loop for controlled drug delivery devices or other therapeutic devices.

Off line analysis of the characteristic of interest in the fluid is performed in an adapted standard body fluids analyzer, providing an accurate result without the need for invasive extraction of the body fluids like blood.

Embodiments of the present invention are suitable for humans, including infants, disabled patients, patient suffering from needles fear and patients that need frequent body fluids sampling (like blood glucose), mass monitoring and ordinary patients asking to spare the inconvenience involved in the venupuncture process. Additionally, embodiments of the present invention are suitable in veterinary medicine.

The use of blood and oral fluid sampling in veterinary medicine presents several challenges:
i. there is a multitude of animals of different size and character, and there are anatomical, physiological and behavioral constrains particular to different animals;
ii. some farm managers rely on little direct contact with the herds for many weeks or months; and
iii. the animals cannot actively cooperate in consuming the drug or in complying with a specific regimen.

Nonetheless, automatic, autonomous sampling device may allow monitoring (and logging) of the animals health status.

Thus, the present invention successfully addresses the shortcomings of the presently known configurations by providing controlled-specimens-sampling oral devices, which are implanted or inserted into an oral cavity, built onto a prosthetic tooth crown, a denture plate, braces, a dental implant, or the like. The devices are replaced as needed. The controlled oral specimens sampling may be passive, or electro-mechanically controlled, for a high-precision, intelligent, sampling. Additionally, the controlled sampling may be any one of the following: sampling in accordance with a preprogrammed regimen, sampling at a controlled rate, delayed sampling, pulsatile sampling, chronotherapeutic sampling, closed-loop sampling, responsive to a sensor's input, sampling on demand from a personal extracorporeal system, sampling regimen specified by a personal extracorporeal system, sampling on demand from a monitoring center, via a personal extracorporeal system, and sampling regimen specified by a monitoring center, via a personal extracorporeal system. Specimen sampling in the oral cavity may be assisted or induced by a transport mechanism, such as any one of, or a combination of iontophoresis, electroosmosis, electrophoresis, electroporation, sonophoresis, and ablation. The oral devices require replacement at different intervals of minutes, hours, days, weeks or months, maintain a desired sampling rate in the oral cavity, for diverse periods, and by being automatic, ensure adherence to a prescribed sampling regimen. The oral devices and methods for controlled specimen sampling apply to humans and animals.

According to one aspect of the present invention, there is provided a device for sampling of oral fluids and blood, comprising:

a reservoir for collection of the specimens; and

an electronic sampling mechanism,

the device being adapted for insertion to an oral cavity of a subject.

According to one aspect of the present invention, the specimens are sampled through a combination of electronic and passive mechanisms.

According to an additional aspect of the present invention, the device is adapted to be removably inserted to the oral cavity of the subject.

According to an alternative aspect of the present invention, the device is adapted to be permanetly inserted to the oral cavity of the subject.

According to an additional aspect of the present invention, the device is adapted to be permanently inserted to the oral cavity of the subject, and the device further includes a removable component, which houses at least one of the specimens' reservoir and the power source.

According to an additional aspect of the present invention, the electronic sampling mechanism further includes:

a control unit, for controlling the sampling rate and timing;

an electromechanical sampling mechanism, which opens to allow the passage of the fluids, responsive to commands from the control unit; and

a power source, for powering the control unit and electromechanical sampling mechanism.

According to one aspect of the present invention, the electromechanical sampling mechanism includes a rotor.

According to an additional aspect of the present invention, the control unit is selected from the group consisting of a dedicated electronic circuitry, a processor, an ASIC, a microcomputer and discrete components.

According to an additional aspect of the present invention, the device for specimens sampling further includes a timing device, selected from the group consisting of a timer, a clock, a calendar, and a combination thereof.

According to an additional aspect of the present invention, the device further includes at least one local sensor, integrated with the device.

According to an additional aspect of the present invention, the device further includes at least two local sensors, integrated with the device.

According to an additional aspect of the present invention, the at least one local sensor is a physiological sensor.

According to an additional aspect of the present invention, the local physiological sensor is selected from the group consisting of a sensor for electrolyte concentration in oral fluids, a sensor for glucose concentration in oral fluids, a sensor for a metabolite concentration in oral fluids, a sensor for the pH level in oral fluids, a sensor for the temperature in the oral cavity, a heartbeat sensor, a heart rate sensor, and a snoring sensor.

According to an additional aspect of the present invention, the at least one local sensor is a status sensor, for ensuring that the device is in proper operating condition.

According to an additional aspect of the present invention, the local status sensor is selected from the group consisting of a sensor for specimen quantity in the reservoir, a sensor for fluid flow rate, a sensor for power source condition, and a sensor for short-circuit detection.

According to an additional aspect of the present invention, the device further includes at least one communication component, selected from the group consisting of a receiver, a transmitter, and a transceiver.

According to an additional aspect of the present invention, the communication component provides communication with a personal extracorporeal system.

According to an additional aspect of the present invention, the personal extracorporeal system is selected from the group consisting of a remote control unit, a computer system, a telephone, a mobile phone, a palmtop, a PDA, a laptop, and a combination thereof.

According to an additional aspect of the present invention, the personal extracorporeal system is adapted to provide communication between the device and a monitoring center.

According to an additional aspect of the present invention, the communication component provides communication with at least one remote sensor.

According to an additional aspect of the present invention, the remote sensor is selected from the group consisting of a sensor for metabolite concentration in oral fluids, a sensor for glucose concentration in the blood and oral fluids, a sensor for a metabolite concentration in the blood, a sensor for an electrolyte concentration in the blood and oral fluids, a sensor for oxygen level in the blood and oral fluids, a sensor for the pH level in the blood and oral fluids, a sensor for glucose concentration in the interstitial fluid, a sensor for a metabolite concentration in the interstitial fluid, a sensor for an electrolyte concentration in the interstitial fluid, a sensor for oxygen level in the interstitial fluid, a sensor for the pH level in the interstitial fluid, a temperature sensor, a heartbeat sensor, a heart rate sensor, a blood pressure sensor, and sensors for the presence of food, alcohol or tobacco in the mouth.

According to an additional aspect of the present invention, the device further includes at least one fluid, electrolyte and metabolite-transfer component for increased fluid transfer through a biological barrier, by a process selected from the group consisting of iontophoresis, electroosmosis, electrophoresis, electroporation and sonophoresis.

According to an additional aspect of the present invention, the specimens sampling mechanism provides the controlled sampling in a manner selected from the group consisting of sampling in accordance with a preprogrammed schedule, sampling at a controlled rate, delayed sampling, pulsatile sampling, chronotherapeutic sampling, closed-loop sampling, responsive to a sensor's input, sampling on demand from a personal extracorporeal system, sampling in accordance with a schedule specified by a personal extracorporeal system, sampling on demand from a monitoring center, via a personal extracorporeal system, and sampling in accordance with a schedule specified by a monitoring center, via a personal extracorporeal system.

According to an additional aspect of the present invention, the device further includes at least two specimens' reservoirs.

According to an additional aspect of the present invention, each of the at least two specimens reservoirs is controlled independently of the other.

According to an additional aspect of the present invention, the device is mounted on a dental implement, designed for the oral cavity of the subject.

According to an additional aspect of the present invention, the device is mounted on a clasps attached to a tooth or several teeth, designed for the oral cavity of the subject.

According to an additional aspect of the present invention, the dental implement is selected from the group consisting of a prosthetic tooth crown, a dental bridge, a dental three-unit bridge, dental implant, partial dentures, full dentures, braces, a molar band, a night guard, and a mouth guard.

According to an alternative aspect of the present invention, the device is mounted on an anchor that may be secured to the oral mucosa or the jawbone.

According to an alternative aspect of the present invention, the device is anchor-free, and is directly implanted into a tissue.

According to an additional aspect of the present invention, the device is adapted for buccal sampling.

According to an additional aspect of the present invention, the device is adapted for sublingual sampling.

According to an additional aspect of the present invention, the device is adapted for labial mucosa sampling.

According to an additional aspect of the present invention, the device is adapted for soft-palatal sampling.

According to an additional aspect of the present invention, the device is adapted for salivary, crevicular or sputum fluid sampling.

According to an additional aspect of the present invention, the device is adapted for oral mucosal specimen sampling.

According to an additional aspect of the present invention, the device is adapted for insertion in a mouth of a human.

According to an alternative aspect of the present invention, the device is adapted for insertion in a mouth of an animal.

According to another aspect of the present invention, there is provided a method of controlled specimen sampling, comprising a reservoir containing the collected specimens and an electronic specimen sampling mechanism for controllably sampling the specimens.

According to another aspect of the present invention, there is provided a device for controlled specimens sampling, comprising:

a reservoir containing the collected specimens; and

a dental implement, designed to be inserted to the oral cavity of a subject, and adapted for supporting the specimen reservoir.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the patent specification, including definitions, will control. In addition, the materials, methods, and examples are illustrative only and not intended to be limiting.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1 is a cross-sectional view of a tooth, as known;

FIGS. 2A-2G schematically illustrate the steps in root canal therapy, as known;

FIGS. 3A-3F schematically illustrate the application of a dental bridge, as known;

FIGS. 4A-4C schematically illustrate the application of a dental implant, as known;

FIGS. 5A-5C schematically illustrate the dentures, as known;

FIGS. 6A-6C schematically illustrate the application of a dental crown, as known;

FIGS. 7A-7C schematically illustrate the braces, as known;

FIGS. 8A-8D schematically illustrate dental bridges, which include devices for specimens sampling, in accordance with preferred embodiments of the present invention;

FIGS. 9A-9B schematically illustrate electronic devices for specimens sampling, in accordance with preferred embodiments of the present invention;

FIG. 10 schematically illustrates electromechanical specimens sampling mechanisms, operative with the electronic devices for specimens sampling;

FIGS. 11A-11D schematically illustrate dentures, which include at least one device for specimens sampling, in accordance with another preferred embodiment of the present invention;

FIGS. 12A-12H schematically illustrate dental braces, which include at least one device for specimens sampling, in accordance with another preferred embodiment of the present invention.

FIGS. 13A-13K schematically illustrates, which include at least one method for specimens sampling, in accordance with another preferred embodiment of the present invention.

FIG. 14 schematically illustrates a block diagram, which include at least one method for specimens sampling, in accordance with another preferred embodiment of the present invention.

FIG. 15 schematically illustrates a block diagram, which include at least one method for specimens sampling, and its extracorporal device for communication, control and data logging and/or controlled drug delivery device in which the delivered dosage is influenced by the sensing mechanism in accordance with another preferred embodiment of the present invention.

FIG. 16 schematically illustrates an example of the intraoral device shape, which includes at least one method for specimens sampling, in accordance with another preferred embodiment of the present invention.

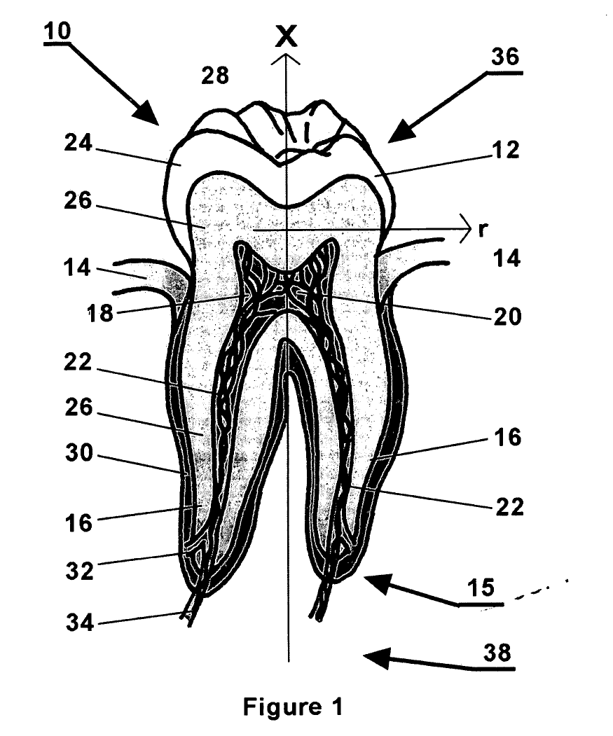

### DESCRIPTION OF THE PREFERRED EMBODIMENTS

The present invention is of oral specimens sampling devices, which are implanted or inserted into an oral cavity, built onto a prosthetic tooth crown, a denture plate, braces, a dental implant, or the like. Specimens sampling may be passive or electro-mechanically controlled, for a high-precision, intelligent sampling. Additionally, the specimens sampling may be any one of the following: sampling in accordance with a preprogrammed regimen, sampling at a controlled rate, delayed sampling, pulsatile sampling, chronotherapeutic sampling, closed-loop sampling, responsive to a sensor's input, sampling on demand from a personal extracorporeal system, sampling regimen specified by a personal extracorporeal system, sampling on demand from a monitoring center, via a personal extracorporeal system, and sampling regimen specified by a monitoring center, via a personal extracorporeal system. Specimens sampling in the oral cavity may be assisted or induced by a transport mechanism, such as any one of, or a combination of iontophoresis, electroosmosis, electrophoresis, electroporation, sonophoresis, and ablation. The oral devices and methods for controlled specimens sampling apply to humans and animals.

Specifically, the present invention relates to body-fluid sensing and monitoring, and to oral devices, mounted on dental implements, configured for sampling and monitoring of blood, saliva, other oral fluids, such as sputum and crevicular fluid, and various other internal body fluids. Additionally, the present invention relates to methods of clinical sampling and analyzing, by the oral devices.

More specifically, the present invention relates to devices and methods for non-invasive (or minimally invasive) blood and oral fluids sampling in a subject, by the collection of blood and oral fluids from the oral tissues and cavity of the subject. Blood and oral fluids are obtained from the tissues using mainly reverse drug delivery techniques such as iontophoresis, sonophoresis, RF microchanneling techniques. The sample is taken out to a sampling analyzer and/or analyzed by built-in sensors and the results are transmitted to an extraoral monitoring device or to a controlled drug delivery system or other therapeutic system, such as an electrostimulator to deliver the appropriate dosage or stimulation based on the sensing results.

Built-in analysis of the characteristic of interest in the fluid is performed in the specified collection device, providing instantaneous results without the manual handling of the fluid sample. The results are transmitted to a monitoring and logging device and/or used to close the loop for controlled drug delivery devices or other therapeutic devices.

Off line analysis of the characteristic of interest in the fluid is performed in an adapted standard body fluids analyzer, providing an accurate result without the need for invasive extraction of the body fluids like blood.

Embodiments of the present invention are suitable for humans, including infants, disabled patients, patient suffering from needles fear and patients that need frequent body fluids sampling (like blood glucose), mass monitoring and ordinary patients asking to spare the inconvenience involved in the venupuncture process. Additionally, embodiments of the present invention are suitable in veterinary medicine.

The principles and operation of the devices and methods according to the present invention may be better understood with reference to the drawings and accompanying descriptions.

Before explaining at least one embodiment of the invention in detail, it is to be understood that the invention is not limited in its application to the details of construction and the arrangement of the components set forth in the following description or illustrated in the drawings. The invention is capable of other embodiments or of being practiced or carried out in various ways. Also, it is to be understood that the phraseology and terminology employed herein is for the purpose of description and should not be regarded as limiting.

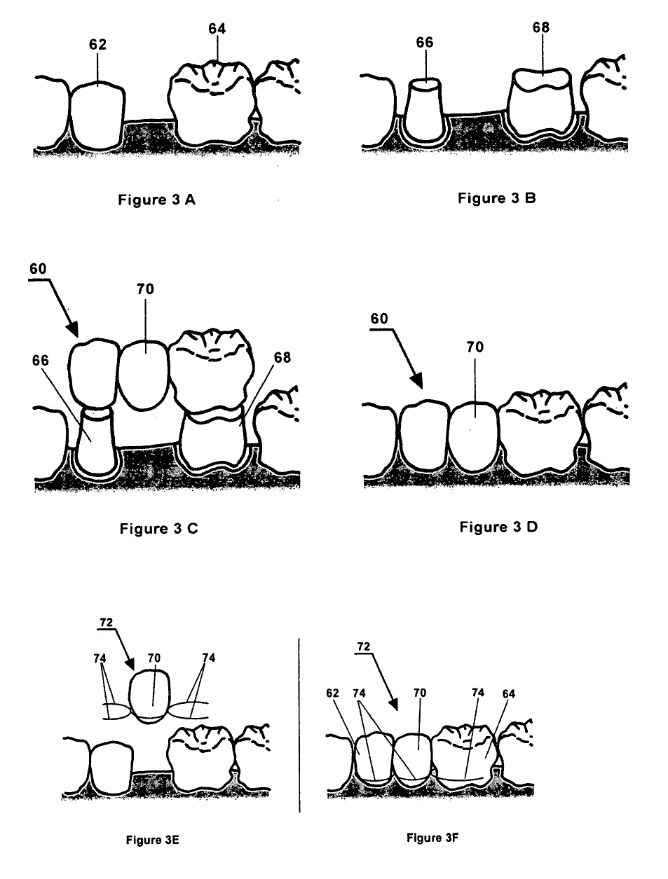

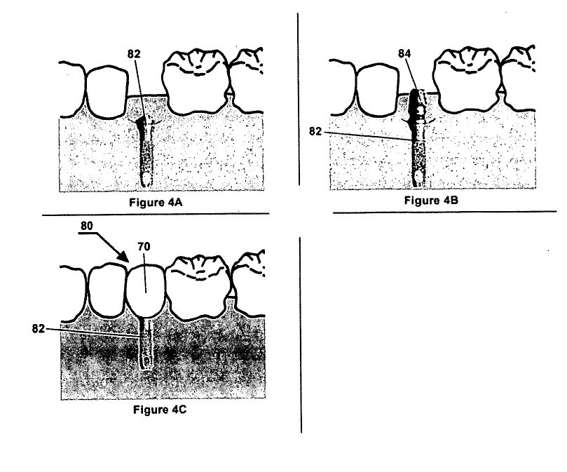

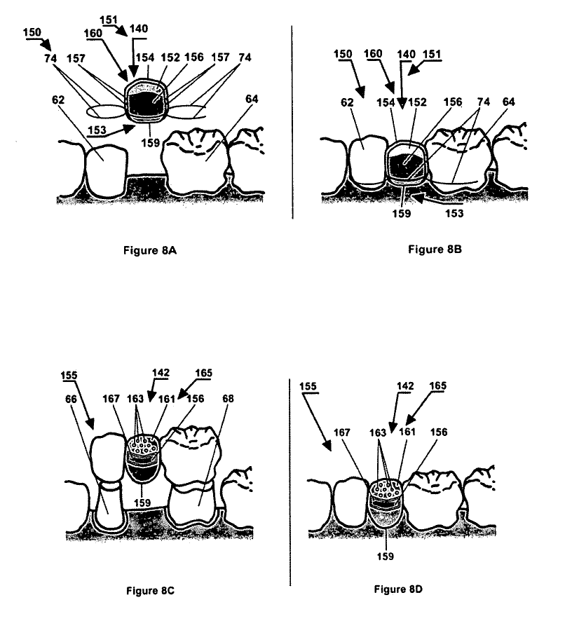

Referring now to the drawings, FIGS. 8A-8B schematically illustrate a device 140, for controlled specimens sampling, mounted on a dental bridge 150, in accordance with a preferred embodiment of the present invention. Preferably, dental bridge 150 is removable, constructed in the manner taught in FIGS. 3E-3F, hereinbelow.

Device 140, for controlled specimens sampling, is designed as a prosthetic tooth crown 160, and mounted on dental bridge 150, for insertion in the gap between teeth 62 and 64, with clamps 74. Preferably, impressions of teeth 62 and 64 and the gap between them have been made, and dental bridge 150 with prosthetic tooth crown 160 are adapted for a specific patient. Prosthetic tooth crown 160 preferably includes a hard outer shell 154, for example, of metal or porcelain, having a coronal side 151 and an apical side 153, wherein the coronal surface is adapted for chewing.

An inner space of prosthetic tooth crown 160 includes a specimen's reservoir 156, in a dosage form adapted for passive, controlled sampling. As used herein, passive specimens sampling relates to controlled sampling, which is not governed by an electronic device. Passive specimens sampling includes for example, the methods of dosage form preparation described hereinbelow, in items 1-14.

Preferably, hard outer shell 154 includes at least one, and preferably several perforations 157 for the specimens sampling. Additionally or alternatively, a semi-pervious membrane 159 may be used, for example on apical side 153. In accordance with the present invention, one or several perforations 157 and (or) semi-pervious membrane 159, may be operative in the controlled sampling of the specimens. Once placed in the oral cavity, the specimens are sampled in a controlled manner, by a natural phenomenon.

Two or more dental bridges 150 may be prepared for a patient, in order to maintain a steady sampling of specimens.

Referring now to the drawings, FIGS. 8C-8D schematically illustrate a device 142, for passive, controlled specimens sampling, mounted on a three-unit bridge 155, analogous to that taught in FIGS. 3A-3D, hereinbelow, in accordance with another preferred embodiment of the present invention.

As seen in FIGS. 3A-3B, hereinbelow, the dentist prepares teeth 62 and 64 on either side of a gap by removing portions of the enamel and dentin, leaving stumps 66 and 68. Impressions or molds of stumps 66 and 68 and of the gap between them are taken for the construction of bridge 155.

As seen in FIGS. 8C-8D, three-unit bridge 155 includes device 142, for passive, controlled specimens sampling, designed as a prosthetic tooth crown 165. Prosthetic tooth crown 165 has a hard outer shell 161, for example, of metal or porcelain, adapted as a chewing surface. Hard outer shell 161 includes a removable component, such as a drawer 167, for refilling, or for replacement. Drawer 167 includes specimens' reservoir 156, in a form adapted for passive, controlled sampling, similar, for example, to that of FIGS. 8A-8B. Preferably, hard outer shell 161 includes at least one, and preferably several or a plurality of perforations 163 for the specimens sampling, or another manner of opening, for the specimens sampling. Additionally or alternatively, semi-pervious membrane 159 may be used.

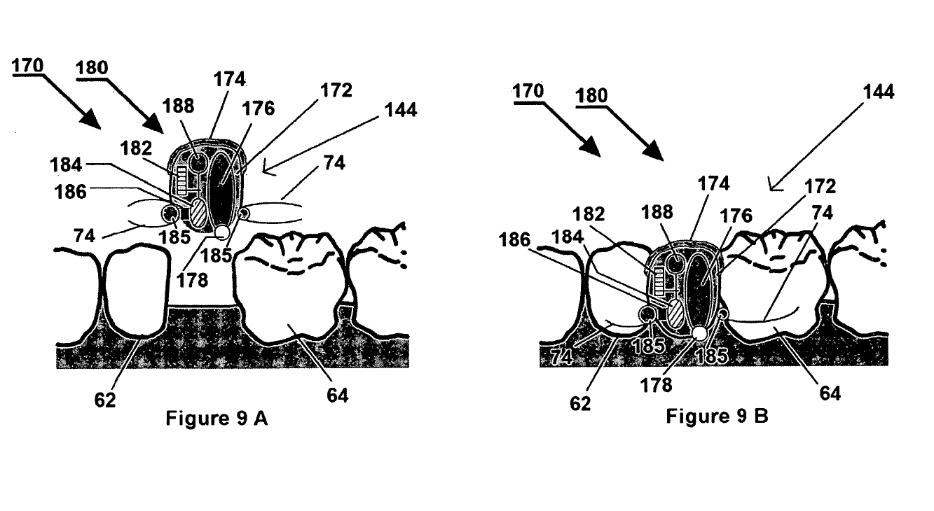

Referring further to the drawings, FIGS. 9A-9C schematically illustrate devices 144 for electronic, controlled specimens sampling, for high-precision, intelligent specimens sampling, in accordance with another preferred embodiment of the present invention.

As seen in FIGS. 9A and 9B, device 144 is mounted on a dental bridge 170. Dental bridge 170 is preferably, removable, constructed in the manner taught in FIGS. 3E-3F, hereinbelow.

Device 144 for electronic, controlled specimens sampling is designed to fit within an inner space of a prosthetic tooth crown 180, for insertion in a gap between teeth 62 and 64, with clamps 74. Preferably, dental bridge 170 is adapted for a specific patient. Prosthetic tooth crown 180 preferably includes a hard outer shell 174, adapted as a chewing surface.

The electronics of device 144 may be encased within filler 172, for example, silicon. Prosthetic tooth crown 180 includes a specimens reservoir 176, having an orifice controlled by an electromechanical sampling mechanism, such as a solenoid 178.

It will be appreciated that specimens reservoir 176 may be in a specimens sampling form for a controlled collection, for example, collection at a controlled rate, delayed collection, and (or) pulsatile collection, which may operate in combination with the electronically controlled sampling.

It will be appreciated that several specimens' reservoirs 176 may be incorporated into a single device 144. Each specimen's reservoir may contain a different specimen, and each specimen may be collected independently, in accordance with a different regimen.

A power source 182 provides prosthetic tooth crown 180 with power. A control unit 184 controls the operation of electromechanical sampling mechanism 178, for the collection of specimens to the oral cavity and (or) oral tissue, in a controlled manner. Control unit 184 may be any one of a dedicated control circuitry 184, a processor 184, an Application Specific Integrated Circuit (ASIC) 184, or a microcomputer 184, as known, and may further include built-in intelligence. A memory unit 186 may be integrated with it. It will be appreciated that control unit 184 may control both the timing for specimens sampling and the sampling rate. It will be appreciated that power source 182 may be any power source, for example, a battery or a solid-electrolyte fuel cell.

Preferably, control unit 184 has a built-in timing device, which preferably includes a timer, a clock and a calendar, and is operative to perform chronotherapy.

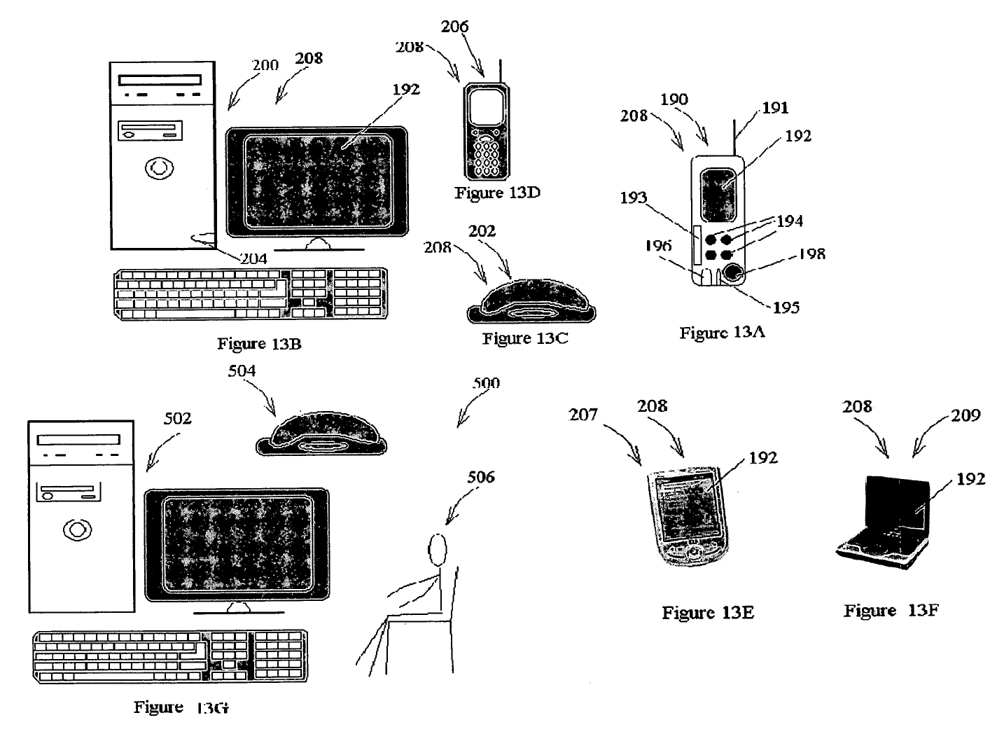

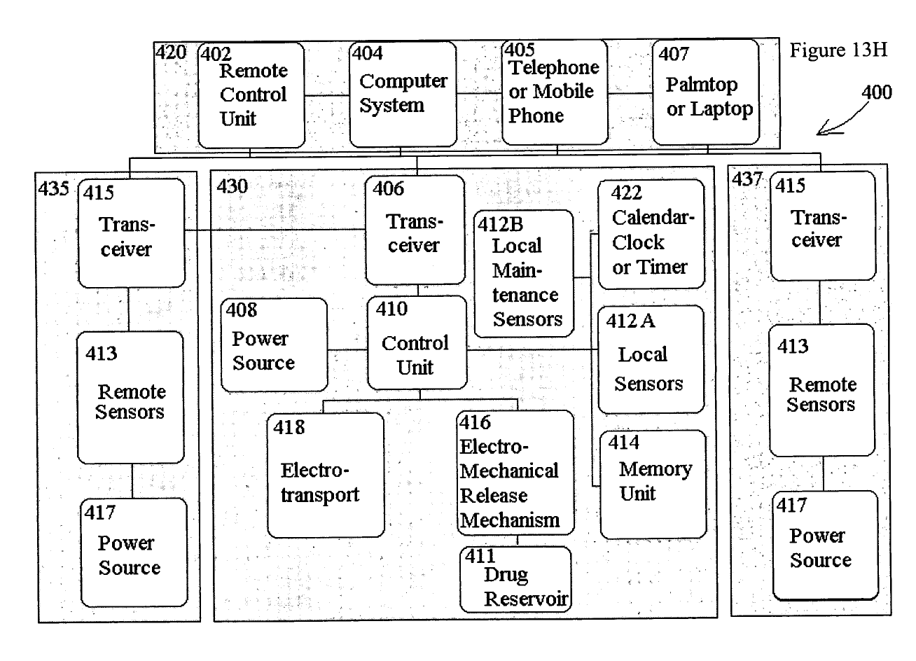

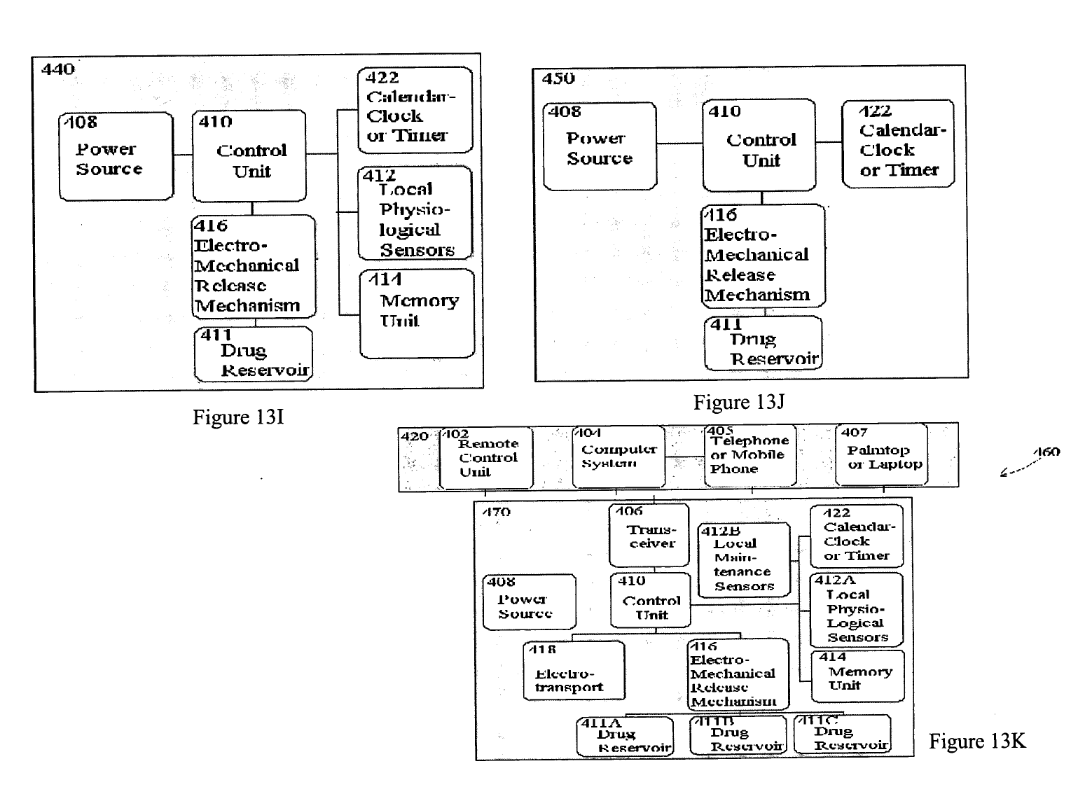

Additionally, a receiver 188, which may further operate as a transceiver, provides communication with a personal extracorporeal system 208, for example, as described in conjunction with FIGS. 13A-13K, hereinbelow. It will be appreciated that a separate transmitter may be used. Transceiver 188 may operate by RF, IR or ultrasound. It may further utilize any one of Bluetooth, Wi-Fi, W-LAN, 802.11, CDMA, GSM protocols. It will be appreciated that other protocols may also be used.

In accordance with the present invention, device 144 for electrically controlled specimens sampling may further include at least one specimens-transfer component for increased specimens transfer through a biological barrier, to enhance buccal, sublingual, labial mucosa and soft-palatal direct sampling. The specimens transfer mechanism may include iontophoresis, electroosmosis, electrophoresis, electroporation, sonophoresis, ablation. The at least one specimens-transfer component may be, for example, at least one electrode or several electrodes, for an electrotransport mechanism including electric ablation, an ultrasound transducer, for sonophoresis, a microwave coil, for microwave ablation, an RF coil, for RF ablation, or a laser diode, for laser ablation, as known. Additionally, a combination of these may be employed. These mechanisms may be controlled by control unit 184. Additionally or alternatively, they may be controlled remotely, by personal extracorporeal system 208 (FIGS. 13A-13K), such as remote-control unit 190, computer system 200, telephone 202, mobile phone 206, palmtop 207, laptop 209, or any other remote-control unit, as known. Additionally or alternatively, they may be controlled by monitoring center 500 (FIG. 13G).

Device 144 may further include at least one and preferably several sensors 185, incorporated to device, and thus termed “local sensors,” to distinguish them from remote sensors, located elsewhere in the body. Local sensors 185 may be divided into two groups:

i. physiological sensors 185, for measuring, for example, an analyte concentration in the saliva, glucose concentration in the saliva, a metabolite concentration in the saliva, an electrolyte concentration in the saliva, the pH level in the saliva, a concentration level of food, alcohol, or tobacco, the temperature in the oral cavity, and any other physiological parameter or parameters, preferably having a bearing on the specimens sampling schedule; and

ii. status sensors 185, for ensuring that the device is in proper operating condition, for example, by measuring the amount of specimens accumulated in the specimens reservoir, the specimens flow rate, the power source condition, a short circuit, or any other information relevant to the proper operation of device 144 for electronic, controlled specimens sampling.

Physiological sensor 185 may be, for example, an electrochemical glucose sensor, such as a enzymatic biosensor taught in www.cfdrc.com/applications/biotechnology/biosensor.html, which utilizes the biospecificity of an enzymatic reaction, along with an electrode reaction that generates an electric current or a potential difference for quantitative analysis. The enzymatic oxidation of glucose produces hydrogen peroxide, which in turn generates electrons by electrode reaction. The current density is used as a measure of glucose in a sample, for example, in interstitial fluid.

Additionally or alternatively, glucose levels may be monitored for example, as taught by U.S. Pat. No. 6,201,980, to Darrow, et al., dated Mar. 13, 2001, entitled, “Implantable medical sensor system,” whose disclosure is incorporated herein by reference. Darrow, et al. disclose an implantable chemical sensor system for medical applications, which permits selective recognition of an analyte using an expandable biocompatible sensor, such as a polymer, that undergoes a dimensional change in the presence of the analyte. The expandable polymer is incorporated into an electronic circuit component that changes its properties (e.g., frequency) when the polymer changes dimension. As the circuit changes its characteristics, an external interrogator transmits a signal transdermally to the transducer, and the concentration of the analyte is determined from the measured changes in the circuit. The implantable chemical sensor system may be used for minimally invasive monitoring of blood glucose levels or interstitial fluid glucose levels in diabetic patients.

Additionally or alternatively, physiological sensors 185 may be, for example, as taught by U.S. Pat. No. 6,058,331, to King, dated May 2, 2000, and entitled, “Apparatus and method for treating peripheral vascular disease and organ ischemia by electrical stimulation with closed loop feedback control,” whose disclosure is incorporated herein by reference. King discloses techniques for therapeutically treating peripheral vascular disease, wherein a sensor is employed for sensing the extent of blood flow in a patient's limb or ischemic pain and generating a response, based on the sensor's reading.

Alternatively, physiological sensors 185 may be based on Ambri's Ion Channel Switch (ICS™) technology of biosensors of a self assembling synthetic bio-membrane, as described in www.ambri.com/Content/display.asp?screen=174. It is one of the world's first true ‘bio-nano’ devices. Ambri has built a biological switch: a membrane, which can detect the presence of specific molecules and signal their presence by triggering an electrical current. This device—the Ambri Ion Channel Switch (ICS™) Biosensor—is a two molecular layer self assembled membrane based on the ion channel gramicidin.

As taught by PCT publication WO 0174446, to Karachurov, a plurality of miniature sensors of a same type may be employed, to increase the accuracy of the measurements. Additionally or alternatively, sensors of different types may be used. Furthermore, several sensor modules 185 may be employed, at different locations in the body.

Device 144 may further include at least one, and preferably several remote physiological sensors 185, implanted or otherwise placed elsewhere in the body, each having its own power supply and transmitter or transceiver. Additionally or alternatively, a remote sensor module 185 of several physiological sensors, possibly of different types, may be employed, wherein the several sensors share a power supply, a transmitter or transceiver, and possibly a control unit. The remote sensor module may further include a remote status sensor 185, for reporting the remote-sensor power source condition.

Examples of remote physiological sensors 185 may include a sensor for analyte concentration in the blood, a sensor for glucose concentration in the blood, a sensor for a metabolite concentration in the blood, a sensor for an electrolyte concentration in the blood, a sensor for oxygen level in the blood, a sensor for the pH level in the blood, a sensor for alcohol level in the blood, a sensor for specimens concentration in the interstitial fluid, a sensor for glucose concentration in the interstitial fluid, a sensor for a metabolite concentration in the interstitial fluid, a sensor for an electrolyte concentration in the interstitial fluid, a sensor for oxygen level in the interstitial fluid, a sensor for the pH level in the interstitial fluid, a sensor for alcohol level in the blood, a sensor for specimens concentration in the sweat, a temperature sensor, a heartbeat sensor, a blood pressure sensor, a heart rate sensor, and a snoring sensor.

Remote sensors 185 may be intracorporeal, implanted under the skin, for example, in the chest or under the arm, for measuring, for example, interstitial fluid specimens' concentration level, interstitial fluid glucose level, tissue temperature, blood pressure, and heart rate. Additionally or alternatively, remote sensors 185 may be intracorporeal, implanted on stents, in blood vessels, for measuring, for example, blood specimens' concentration level, blood glucose level, or blood oxygen level.

Additionally or alternatively, remote sensors 185 may be extracorporeal, for example, attached to the skin. The extracorporeal sensors may include piezoelectric patches that may be attached to the skin, by adhesives, for measuring heart rate, patches for measuring body temperature, and (or) sensors that measure concentration levels of the specimens, or of other chemicals, such as glucose, in the sweat.

For example, extracorporeal, remote sensors 185 may be similar to those taught by Lin, G., and Tang, W., “Wearable Sensor Patches for Physiological Monitoring,” NASA's Jet Propulsion Laboratory, Pasadena, Calif., which may be found at www.nasatech.com/Briefs/Feb00/NPO20651.html, or in NASA Tech Briefs: NPO-20651, which may be obtained from Technology Reporting Office, JPL, Mail Stop 122-116, 4800 Oak Grove Drive, Pasadena, Calif. 91109, (818) 354-2240. The wearable sensor patches, formed as miniature biotelemetric units, may be employed for measuring temperature, heart rate, blood pressure, and possibly other physiological parameters. The sensor patches are designed small and may be mass-produced inexpensively by use of state-of-the-art techniques for batch fabrication of integrated circuits and microelectromechanical systems. Each patch may be a few centimeters on a side, comparable in size to an ordinary adhesive bandage. The patch may even be held on the wearer's skin by the same adhesive as that used on bandages. The patch may contain a noninvasive microelectromechanical sensor integrated with electronic circuitry operative to process the sensor output and transmit a radio signal modulated by the processed sensor output.

As for the local sensors, a plurality of miniature sensors of a same type may be employed, to increase the accuracy of the measurements. Additionally or alternatively, sensors of different types may be used. Furthermore, several sensor modules 185 may be used, at different locations in the body.

Communication between remote sensors 185 and prosthetic tooth crown 180 of device 144 is preferably by ultrasound, but may be by IR or RF, and may employ communication protocols, such as any one of Bluetooth, Wi-Fi, W-LAN, 802.11, CDMA, RFID, GSM protocols. It will be appreciated that other protocols may also be used. Additionally or alternatively, remote sensors 185 may communicate with one or more personal extracorporeal systems 208, as described in conjunction with FIGS. 13A-13K, hereinbelow, preferably by IR or RF, and may employ communication protocols, such as any one of Bluetooth, Wi-Fi, W-LAN, 802.11, CDMA, RFID, GSM protocols. It will be appreciated that other protocols may also be used. Communication may be on a continuous basis, at intervals, in reply to interrogation, or when a sudden change in a measured physiological parameter is observed.

In accordance with some embodiments, the remote sensors do not have power sources, but respond to interrogation, which further provides them with power for measuring and responding, as known, for example, as described in any one of U.S. patent application No. 20010026111, to Doron et al., “Acoustic biosensor for monitoring physiological conditions in a body implantation site,” U.S. Pat. No. 6,140,740 to Porat, et al “Piezoelectric transducer,” U.S. Pat. No. 6,277,078 to Porat, et al, “System and method for monitoring a parameter associated with the performance of a heart,” and U.S. Pat. No. 6,237,398 to Porat, et al., “System and method for monitoring pressure, flow and constriction parameters of plumbing and blood vessels,” all of whose disclosures are incorporated herein by reference.

It will be appreciated that device 144 may also be a self-contained system, and operate without an extracorporeal system or any remote control.

It will be appreciated that prosthetic tooth crown 180 may also be designed on a three-unit bridge, in a manner analogous to prosthetic tooth crown 165 of FIGS. 8C-8D, wherein parts that need replacement, such as specimens reservoir 176 and possibly also power source 182 are located in a drawer, analogous to drawer 167 there.

As seen in FIG. 9C, device 144 for electronic, controlled specimens sampling, is designed as a dental-implant-and-prosthetic-tooth-crown 210. Device 144 for electronic, controlled specimens sampling has a permanent portion 220, located in the post and a removable portion 230, in the crown. Removable portion 230, in the crown of device 144, includes a specimen's reservoir 216, whose specimens sampling is controlled by an electromechanical sampling mechanism 218. A power source 222 provides power. These are encased within filler 212, for example silicon. A hard shell 214 provides the chewing surface. Preferably, impressions have been taken so that removable portion 230 is adapted for a specific patient. Additionally, two or more removable portions 230 may be made, so that one is in operation while the other is being refilled.

Permanent portion 220, in the post, may include a control unit 224, such as a processor 224, for controlling the operation of electromechanical sampling mechanism 218, preferably also a memory unit 226, and a transmitter-receiver 228. At least one sensor 215 may be located on the interface between the post and the crown, and may be attached to either. Alternatively, at least one sensor 215 may be located within the post or within the crown. Alternatively, the sensor or sensors may be located elsewhere in the body. Electro-mechanical sampling mechanism 218 may be located in the post or in the crown of device 144.

The operation of the present embodiment is similar to that of the embodiment of FIGS. 9A-9B, save for the advantage that only the portions of the electronic device that need replacement, namely the specimens reservoir and the power source, are adapted for removing.

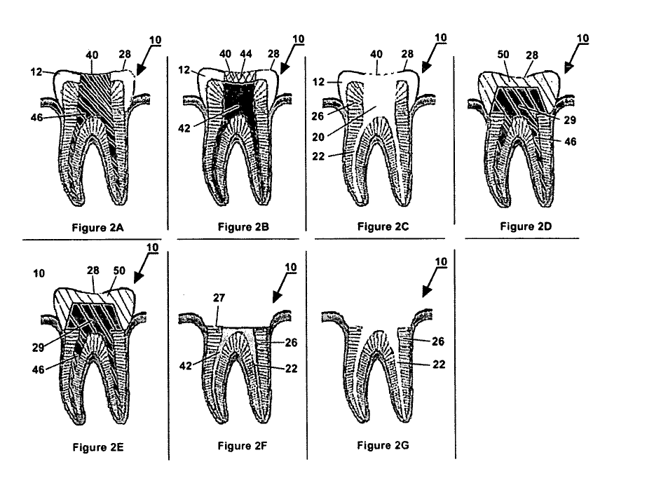

It will be appreciated that a similar construction of a permanent portion and a removable portion may be used in conjunction with a root canal (FIGS. 2A-2G). The permanent portion may be located in the canal, and the removable portion may be located in the crown.

It will be appreciated the crown of device 144 may also be designed in a manner analogous to prosthetic tooth crown 165 of FIGS. 8C-8D, wherein parts that need replacement, such as specimens reservoir 176 and possibly also power source 182 are located in a drawer, analogous to drawer 167 there.

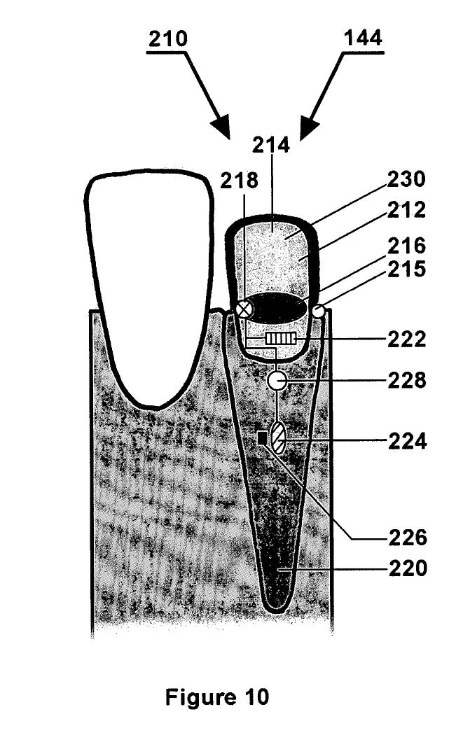

Referring further to the drawings, FIGS. 10A-10F schematically illustrate electromechanical sampling mechanisms 178, operative with the electronic devices for controlled specimens sampling of FIGS. 9A-9C, in accordance with embodiments of the present invention.

As seen in FIGS. 10A-10B, electromechanical sampling mechanism 178 may be designed as a Vane engine 175A, having a housing 171, a rotor 173, an inlet 177 and an outlet 179, wherein inlet 177 is in communication with specimens' reservoir 176, and outlet 179 leads to the oral cavity. As rotor 173 rotates in the direction of an arrow 169, inlet 177 opens, allowing specimens from specimens' reservoir 176 to enter an inner cavity 141 of engine 175A, and be pushed out through outlet 179.

It will be appreciated that inner cavity 141 may be designed so as to avoid specimens compression during the cycle.

In accordance with the present invention, Device 144 (FIGS. 9A-9C) for electronic, controlled specimens sampling may include one or several specimens' reservoirs 176, each in communication with one inlet 177 and one rotor arrangement. Control unit 184 (FIGS. 9A-9C) may translate the amount of specimens to be collected into specimens reservoir 176 to a number of rotor revolutions, so that, with each revolution, a predetermined amount of specimens is issued to the specimens reservoir and collected from the oral cavity.

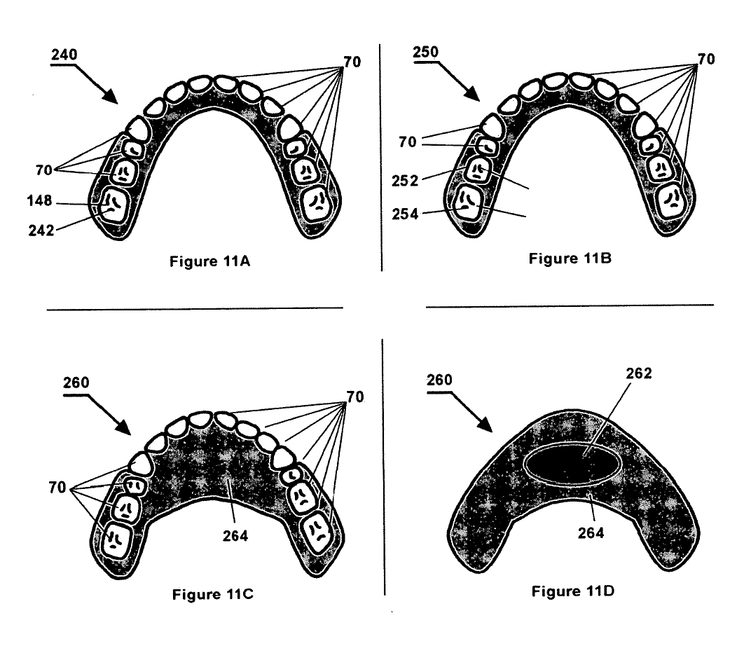

Referring further to the drawings, FIGS. 11A-11D schematically illustrate full dentures, which include at least one device for controlled specimens sampling, in accordance with another preferred embodiment of the present invention. It will be appreciated that partial dentures may similarly be used.

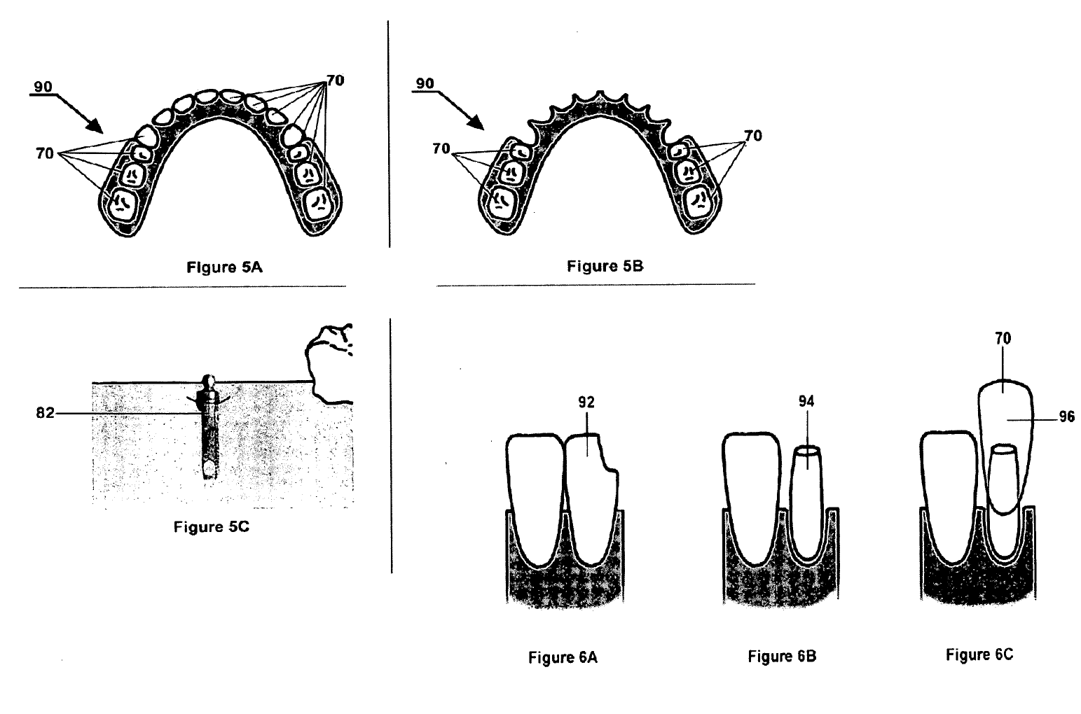

As seen in FIG. 11A, dentures 240 includes a plurality of prosthetic tooth crowns 70, as taught in conjunction with FIGS. 5A-5C, hereinbelow. Additionally, dentures 240 include a device 148 for controlled specimens sampling, designed as a prosthetic tooth crown 242. Prosthetic tooth crown 242 may be adapted for passive controlled specimens sampling, as taught in conjunction with FIGS. 8A-8D. Alternatively, prosthetic tooth crown 242 may be adapted for electronically controlled specimens sampling, as taught in conjunction with FIGS. 9A-9B, and preferably operate with any one of or a combination of personal extracorporeal systems 208, described hereinbelow, in conjunction with FIGS. 13A-13K, and with a monitoring center 500 described hereinbelow, in conjunction with FIG. 13G.

As seen in FIG. 11B, dentures 250 includes a plurality of prosthetic tooth crown 70, as taught in conjunction with FIGS. 5A-5C, hereinbelow. Additionally, dentures 250 include devices 147 and 149, designed as prosthetic tooth crowns 252 and 254, for controlled specimens sampling. These may be adapted for passive controlled specimens sampling, as taught in conjunction with FIGS. 8A-8D, or for electronically controlled specimens sampling, as taught in conjunction with FIGS. 9A-9B, and preferably operate with any one of or a combination of personal extracorporeal systems 208, described hereinbelow in conjunction with FIGS. 13A-13K, and with monitoring center 500, described hereinbelow, in conjunction with FIG. 13G.

Additionally, more than two prosthetic tooth crowns for controlled specimens sampling may be employed.

Alternatively, prosthetic tooth crowns 252 and 254 may form a single device for electronically controlled specimens sampling, wherein prosthetic tooth crown 252 may form a removable portion, which includes the specimens reservoir and power source, which must be replaced periodically, while prosthetic tooth crown 254 may include the permanent components, as taught in conjunction with FIG. 10, hereinbelow.

FIGS. 11C and 11D illustrate front and back sides of full dentures 260, which include a plate 264, which may be fitted under the tongue, for bottom dentures, or against the roof of the mouth, for top dentures. The backside (FIG. 11D) further includes a device 262, for controlled specimens sampling. In this manner, soft-palatal and (or) sublingual, labial mucosa and buccal administration may be enhanced. The advantage of these types of sampling is that they lead to direct collection from the blood stream.

Device 262 for controlled specimens sampling may be passive or electronically controlled.

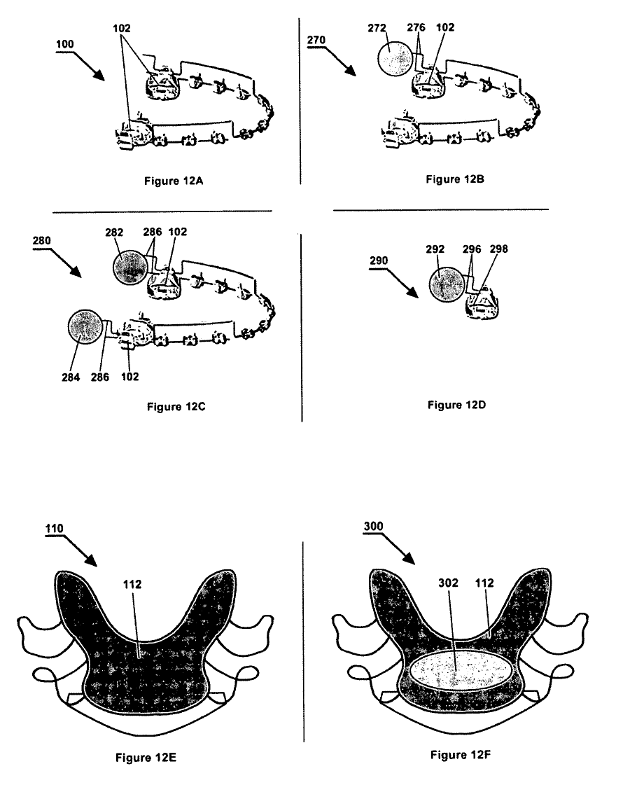

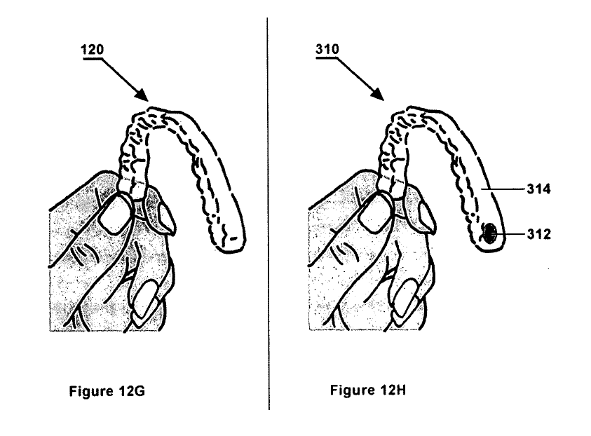

Referring further to the drawings, FIGS. 12A-12H schematically illustrate dental braces, which include at least one device for controlled specimens sampling, in accordance with another preferred embodiment of the present invention.

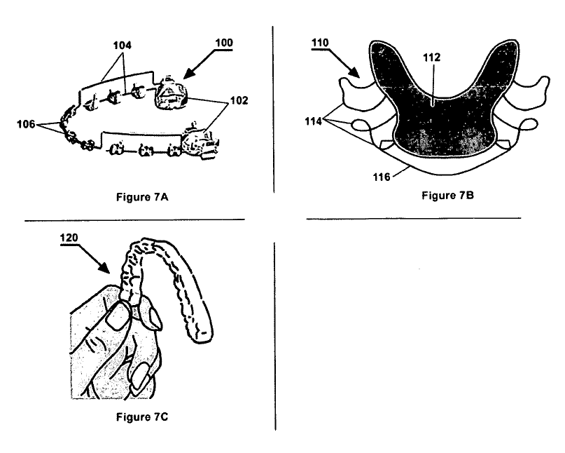

While FIG. 12A schematically illustrates conventional braces 100, having molar bands 102, as taught in conjunction with FIG. 7A, hereinbelow, FIG. 12B illustrates braces 270, which include a device 272 for controlled specimens sampling, in accordance with a preferred embodiment of the present invention. Device 272 is attached to molar bands 102 with wires 276.

Additionally, FIG. 12C illustrates braces 280, which include devices 282 and 284, for controlled specimens sampling, in accordance with a preferred embodiment of the present invention. Devices 282 and 284 are attached to molar bands 102 with wires 286. Additional devices may similarly be employed.

Furthermore, FIG. 12D illustrates an arrangement 290, in which a device 292 for controlled specimens sampling is attached to a molar band 298, with wires 296, in accordance with a preferred embodiment of the present invention.

While FIG. 12E schematically illustrates conventional braces 110, having a plate 112, as taught in conjunction with FIG. 7B, hereinbelow, FIG. 12F illustrates braces 300, which include a device 302 for controlled specimens sampling, arranged on the back side of plate 112, in accordance with a preferred embodiment of the present invention. Thus, device 302 is adapted for enhanced buccal and sublingual administration.

While FIG. 12G schematically illustrates conventional invisible braces 120, as taught in conjunction with FIG. 7C, hereinbelow, FIG. 12H illustrates braces 310, which include a device 312 for controlled specimens sampling, arranged on an added invisible portion 314. In a similar manner, a mouth guard or a night guard may be used, for attaching a device for controlled specimens sampling.

It will be appreciated that since braces are generally employed by children whose wisdom teeth have not yet emerged, the space generally occupied by the wisdom teeth may be used for the extensions shown in FIGS. 12B-12D and 12H.

Devices 272, 282, 284, 292, 302 and 312 for controlled specimens sampling may be passive or electronically controlled.

For optimal placement and (or) anchoring of a device for controlled specimens sampling in an oral cavity of a person, in accordance with the present invention, a dentist may examine the mouth of the person. If the patient has a dental implement, such as a crown, a prosthetic tooth crown, a bridge, dentures, braces, a night guard or a mouth guard, any one of these may be replaced with devices in accordance with the present invention. Alternatively or additionally, the patient may be in need of a dental implement, such as a crown, a prosthetic tooth crown, a bridge, dentures, braces, a night guard or a mouth guard, the needed implement may be prepared so as to include a device in accordance with the present invention. Alternatively or additionally, a wisdom tooth may be missing either because it has not yet emerged, or because it has been extracted, and that space may be used for a device in accordance with the present invention, for example, attached to a molar band, as taught in conjunction with FIG. 12D. Alternatively or additionally, a device may be mounted on a braces plate, even where braces need not be used, for dental reasons, as taught in conjunction with FIG. 12F. Alternatively or additionally, a device may be mounted on a night guard or a mouth guard, even where it need not be used for dental reasons. It will be appreciated that a combination of the above may be used.

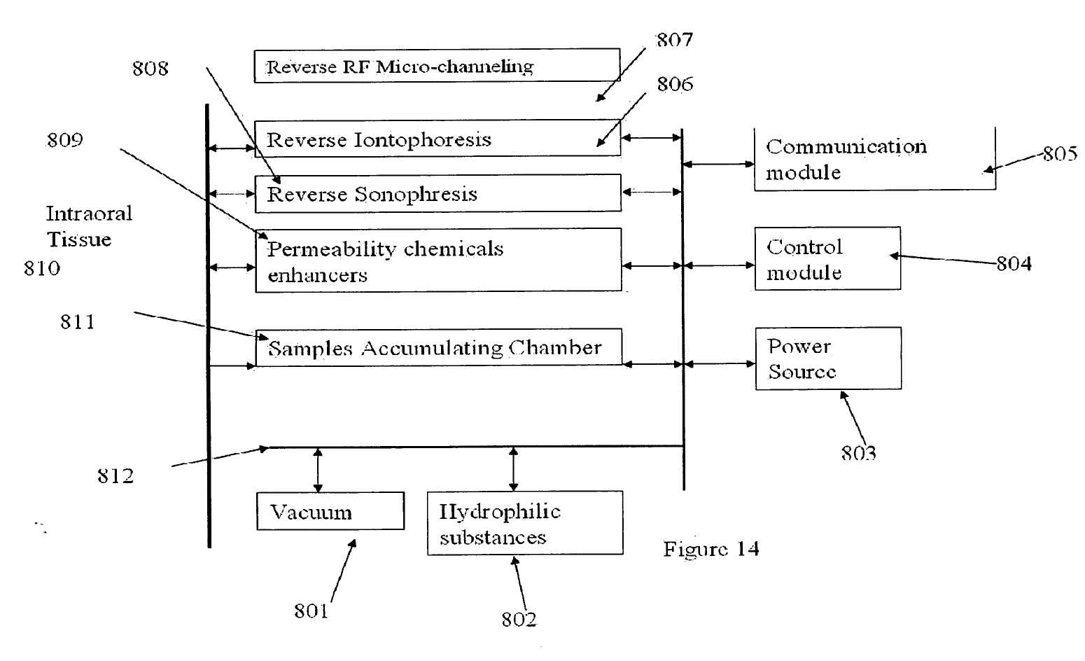

FIG. 14 depicts a typical system block diagram. The final device may exclude some blocks or include any combination thereof. Device 801—generates a vacuum in the samples accumulation chamber directing the flow inwards toward the chamber. Device 802 includes an add-on hydrophilic substance to assist in the direction and accumulation of fluids toward the chamber. Device 803 a power source, batteries (primary, secondary, paper type). Device 804 the electronic based control module based on a microprocessor, ASIC, discrete ICs or any combination thereof. Device 805—communication module for bi-directional or uni-directional communication from the device to the extraporal devices. Communication is based on Infra Red or radio frequency using proprietary protocol or standard protocol like Bluetooth, WiFi, RFID or combination thereof. Device 806—reverse iontophoresis applies electrical current to the intraoral tissue. Device 807—reverse RF microchanneling—creates an array of microchannels in the tissue by applying an RF energy. Device 808—reverse sonophoresis—increase the permeability of the tissue by applying a low frequency (20 KHz to 3 MHz) over the tissue. Device 809 includes tissue permeability enhancers chemicals. Item 810 represents the intraoral tissue itself, gingival, buccal, mucosa, sublingual, palate or any combination thereof. Device 811 represents the chamber that accumulate the extracted fluids and substances to be analyzed locally or by external device. Device 812 includes interconnecting bus connecting electronically and mechanicallys the various devices comprising the final element.

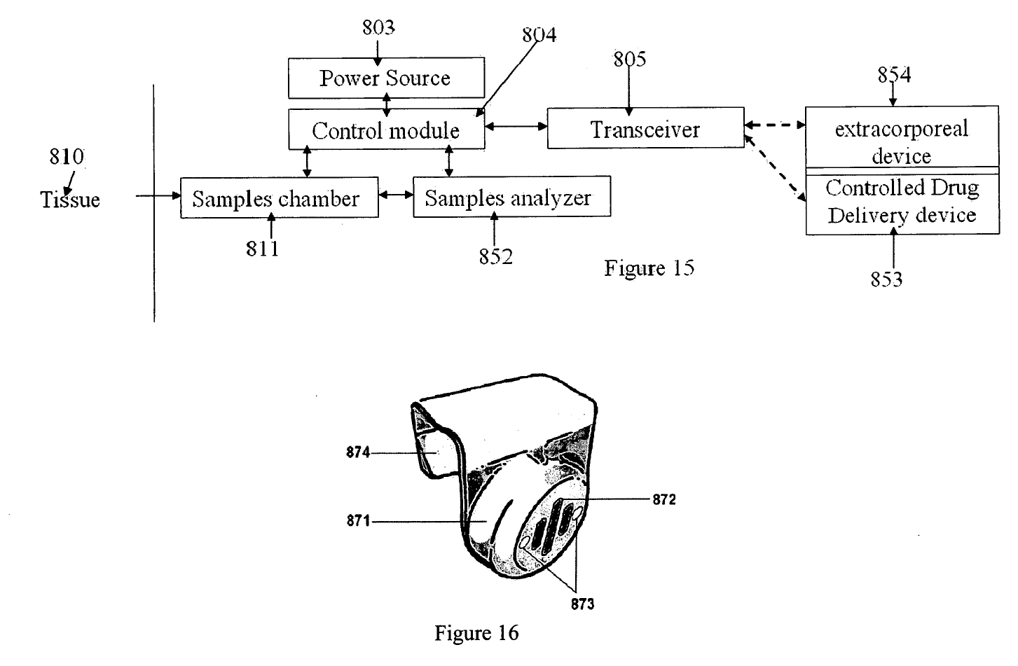

FIG. 15 depicts the basic system block diagram. The final device may exclude some blocks or include any combination thereof. Device 852 is the sample analyzer, analyzing the samples and measure its components in a quantified manner. Device 853—a controlled drug delivery device that releases and delivers the selected drugs in a controlled manner in accordance with the sample sensing results. Device 854—an extraporal device to receive, log, control and communicate with the intraoral sensors.

It will be appreciated that the electronic device for controlled specimens sampling may be mounted on any anchor that may be secured to the oral mucosa or the jawbone. Alternatively, the electronic device for controlled specimens sampling may be directly implanted into a tissue without a specific anchoring element.

FIG. 16 depicts an example of the proposed device. Sub-module 871 is the sampling reservoir. Sub-module 872 are the electrophoresis electrodes. Sub-module 873 inlets for sampled specimens. Sub-module 874 the clasps attached to the teeth. Other elements such as power supply, electronics, communication are embedded inside the device. The inlet opening is facing the buccal tissue and touching it as the mouth is close. The device can be easily inserted or removed.

It will be appreciated that other known anchoring devices, for example as described in U.S. Pat. Nos. 4,175,326, 4,020,558, and 4,681,544 may be used for anchoring devices for controlled specimens sampling, in accordance with the present invention.

In accordance with the present invention, the device for controlled specimens sampling may be implanted or inserted in animals, such as pets, for example, dogs, and cats, farm stock, such as sheep, goats, cows, horses, pigs, and the like, or fowl, such as chickens, ducks, geese, turkeys, and other fowl.

### EXAMPLES

Reference is now made to the following examples, which together with the above description illustrate the invention in a non-limiting fashion.

### Example 1

### Passive, Controlled Specimen Sampling

Device 140, designed as prosthetic tooth crown 160 (FIGS. 8A-8B) for passive, controlled specimen sampling, or another device for passive, controlled specimen sampling may include specimen reservoir 156 and a semi-permeable membrane, which is formed of hydrophobic polymers, such as cellulose acetate, or ethocel, mixed with water soluble additives, such as sugar, PEG'S, and the like. Upon collection, the soluble additives dissolve and a semipermeable membrane is created.

### Example 2

### Delayed, Passive, Controlled Specimen Sampling

Device 140, designed as prosthetic tooth crown 160 (FIGS. 8A-8B) for passive, controlled specimen sampling, or another device for passive, controlled specimen sampling may include several specimen reservoirs 156, wherein a first reservoir is adapted for passive, controlled delivery, for example, by diffusion and erosion, and a second specimen reservoir, which is coated by a special functional coating, designed to halt the sampling into the second reservoir until the first reservoir is full. In this manner, the interval between replacements may be extended.

### Example 3

### Pulsatile, Passive, Controlled Specimen Sampling

Device 140, designed as prosthetic tooth crown 160 (FIGS. 8A-8B) for passive, controlled specimen sampling, or another device for passive, controlled specimen sampling may include a specimen reservoir 156, which includes a multi-layer coating, designed for pulsatile passive controlled sampling, which may be synchronized, for example, with circadian cycles, for a desired chronotherapy.

### Example 4

### Passive, Controlled Specimen Sampling

Prosthetic tooth crown 160 (FIGS. 8A-8B) for passive, controlled specimen sampling, or another device for passive, controlled specimen sampling may include specimen reservoir 156 specimens, incorporated into pellets or minitabs. The collection mechanism is diffusion or erosion. The specimens are replaced once a week.

### Example 5

### Electronic and Passive Controlled Specimen Sampling

Electronic, controlled specimen sampling device 460 (FIG. 14D) may include two or more specimen reservoirs, such as 411A, 411B, and 411C of specimens, incorporated into pellets or minitablets, of a passive, controlled collection, which may last varying periods of time. Upon insertion, electromechanical sampling mechanism 416 opens first specimen reservoir 411A, and controlled collection by diffusion takes place. When the first reservoir 4111A is full, status sensor 412B informs control unit 410, and control unit 410 instructs electromechanical sampling mechanism 416 to open second specimen reservoir 411B. After a certain period of time, second reservoir is full, and third specimen reservoir 411C is opened. In this manner, replacement intervals are extended, as in Example 2, to longer periods of time, depending on the number of specimen reservoirs.

### Example 6

### Electromechanically Controlled Specimen Sampling

In accordance with the present invention, Device 144 (FIGS. 9A-9C) for electronic, controlled specimen sampling may include one or several specimen reservoirs 176, each in communication with one inlet 177 and one rotor arrangement. Control unit 184 (FIGS. 9A-9C) may translate the amount of specimen to be sampled into specimen reservoir 176 to a number of rotor revolutions, so that, with each revolution, a predetermined amount of specimen is issued into the specimen reservoir and collected from the oral cavity.

### Example 7

### Controlled Specimen Sampling for Buccal, Sublingual, Labial Mucosa, and Soft-Palatal Collection, with a Transport Mechanism

The present invention is adapted for specimen absorption from any one of buccal, sublingual, labial mucosa, and (or) soft-palatal sites, which may be further assisted by a mechanism for increased specimen transfer through the biological barrier, for example, by a mechanism such as reverse iontophoresis, electroosmosis, electrophoresis, electroporation, sonophoresis, ablation and RF micro-channeling. Thus, device 144 (FIGS. 9A-9C) for electrically controlled specimen sampling may include at least one specimen-transfer component for increased specimen transfer through a biological barrier. Specifically, the location of the device for controlled specimen sampling may determine the main collection route. For example: sublingual and labial mucosa specimen absorption may be achieved by placing the device for controlled specimen sampling on a bottom-denture plate (FIGS. 11C-11D), or on a bottom-braces place (FIG. 12F), or on a bottom plate of a night guard or a mouth guard; Similarly, soft-palatal specimen absorption may be achieved by placing the device for controlled specimen sampling on a top-denture plate (FIGS. 11C-11D), or on a top-braces place (FIG. 12F), or on a top plate of a night guard or a mouth guard; and buccal specimen absorption may be achieved by placing the device for controlled specimen sampling on a prosthetic tooth crown, (FIGS. 8A-8D).

A transport mechanism, for example, iontophoresis may be incorporated. For example, two biocompatible electrodes may be placed against the buccal tissue, for applying a current of up to 0.5 mA to the buccal surface. The current may be applied in pulses, which may vary by time, or modulating frequency, or as a DC current. (M. B. Delgado-Charro, R. H. Guy; “Transdermal iontophoresis for controlled drug delivery and non-invasive monitoring” October 2001). In a similar manner, the transfer tissue may be any one of sublingual, labial mucosa, and (or) soft-palatal tissue.

Alternatively, or additionally another transport mechanism, for example, sonophoresis may be used. A piezoelectric transducer may be placed against any one of the buccal, sublingual, labial mucosa, and (or) soft-palatal tissue, and adapted to resonate at a frequency of between 20 KHz and 1.5 Mhz. Additionally, the resonation may vary in power, frequency, modulation, duration and pulse width.

### Example 8

### Controlled Specimen Sampling for Veterinary Use

In accordance with the present invention, controlled monitoring, by devices implanted in the mouth cavities of the cows, may be used, to be informed that the plurality of cows ovulate at about the same time, for breeding management. It will be appreciated that many other veterinary uses are possible.

### Example 9

### Chronotherapy Specimen Sampling for Cancer

According to Stehlin [Stehlin I., “A Time to Heal: Chronotherapy Tunes In to Body's Rhythms,” US Food and Drug Administration, www.fda.gov/fdac/features/1997/397 chrono.html], chronotherapy may be useful in the treatment of cancer. Animal studies suggest that chemotherapy may be more effective and less toxic if cancer drugs are administered at carefully selected times. It appears that there may be different chronobiological cycles for normal cells and tumor cells. Thus, if administration of cancer drugs is timed with the chronobiological cycles of tumor cells, it will be more effective against the cancer and less toxic to normal tissues. Thus, any one of device 400, 440, 450, or 460, for electronic, controlled specimen sampling to synchronize the therapy (FIGS. 14A-14D), may be pre-programmed for clock operated specimen sampling, for example, of chemotherapy, for chronotherapy.

By using any one of device 400, 440, 450, or 460, for electronic, controlled specimen sampling (FIGS. 14A-14D), specimen sampling may be synchronized with either predetermined patterns or real-time measurements of physiological parameters. Thus, the cancer patient will receive the cancer drugs in an effective way, with minimal side effects and waste.

### Example 10

### Chronotherapy and Remote Control Specimen Sampling for Arthritis

According to Stehlin [Stehlin I., “A Time to Heal: Chronotherapy Tunes In to Body's Rhythms,” US Food and Drug Administration, www.fda.gov/fdac/features/1997/397 chrono.html], chronotherapy may be useful in the treatment of arthritis. People with osteoarthritis tend to have less pain in the morning and more at night; while those with rheumatoid arthritis, have pain that usually peaks in the morning and decreases throughout the day. Chronotherapy for all forms of arthritis using NTHEs such as ibuprofen may be timed to ensure that the highest blood levels of the drug coincide with peak pain. Devices 400 or 460, for electronic, controlled specimen sampling (FIGS. 14A and 14D), may be pre-programmed for clock-operated specimen sampling, synchronized to the circadian rhythm of the disease, based on the patient's history, for chronotherapy. Chronotherapy may be supplemented by remote control operation, from personal extracorporeal system 420, preferably by the patient, for example, from remote-control unit 402, palmtop 407, or another remote-control unit, when a patient feels pain.

### Example 11

### Chronotherapy, Remote Control and Sensor-Activated Specimen Sampling for Diabetes

Glucose levels vary throughout the day, to some extent in a cyclic manner. Additionally, there is a rise in glucose level shortly after eating. Devices 400 or 460, for electronic, controlled specimen sampling (FIGS. 14A and 14D), may be pre-programmed for clock operated specimen sampling, synchronized to the circadian rhythm of the glucose, for chronotherapy. Preferably, the synchronization is based on the patient's history of glucose level cyclic variations.

Chronotherapy may be supplemented by remote control operation, from personal extracorporeal system 420, preferably by the patient, for example, from remote-control unit 402, palmtop 407, or another remote-control unit, when a patient is about to eat, since he knows that glucose levels will rise then. Additionally, remote control operation may be performed, responsive to a report from one or several sensors 413, that glucose levels in the blood or in the interstitial fluid have risen. The remote control operation, from personal extracorporeal system 420, may be by the patient, for example, from remote-control unit 402 or palmtop unit 407, upon the patient's seeing the glucose level measurement on display. Additionally or alternatively the patient may forward the measurement to monitoring center, for example, via remote-control unit 402 or palmtop unit 407, or another remote-control unit, for the monitoring center's decision, for example of computer, on a specimen sampling schedule.

### Example 12

### Chronotherapy, Remote Control and Sensor-Activated Specimen sampling for Asthma

According to Stehlin [Stehlin I., “A Time to Heal: Chronotherapy Tunes In to Body's Rhythms,” US Food and Drug Administration, www.fda.gov/fdac/features/1997/397 chrono.html], chronotherapy may be useful in the treatment of asthma, since asthmatic patients tend to have attacks during the early hours of the morning, for example, between 3 and 5 AM. Devices 400 or 460, for electronic, controlled specimen sampling may be pre-programmed for clock operated specimen sampling, synchronized to the circadian rhythm of the disease, for chronotherapy, which at times may be further supplemented by remote control operation. Synchronization may be performed on a case by case basis, by preprogramming the device, based on the patient history of the disease. Additionally or alternatively, the specimen sampling rate may be increased a little before the expected time for the attack. Chronotherapy may be supplemented by remote control operation, from personal extracorporeal system 420, preferably by the patient, for example, from remote-control unit 402 or palmtop unit 407, or another remote-control unit, when a patient feels the onset of an attack.

### Example 13

### Sensor-Activated Specimen Sampling for Snoring and Other Sleeping Disorders

For sleeping disorder, a closed loop operation is probably most suitable and device 440 of FIG. 14B may be used. Sensors 412 may be piezo-electric transducers, which sense sound, such as snoring, or heartbeat. The determination and demand for monitoring may be made directly by control unit 410, based on its built-in intelligence and algorithms, for monitoring responsive to the communicated measurements. For snoring the communicated measurement may be the sound of snoring. For insomnia, the communicated measurement may be the rate of heartbeat, indicating whether the patient is asleep or awake.

### Example 14

### Remote Control Specimen Sampling for Mental Diseases

Devices 400 or 460, for electronic, controlled specimen sampling (FIGS. 14A and 14D) may be used by patients suffering from mental conditions such as depression or hypertension. When the situation deteriorates, either the patient, or a caretaker such as a parent may initiate specimen sampling, for example, via remote-control unit 202, palmtop 407, or another remote-control unit. For geriatric patients, suffering from senility or Alzheimer, sensors 412 or 413 may further include a global positioning device, and these may also be mounted on remote-control unit 202, and (or) palmtop 407, or another remote-control unit, for reporting both the location of the patient and of the remote-control unit to the monitoring center.

### Example 15

### Sexual Dysfunction

Devices 400 or 460 for electronic, controlled hormonal monitoring, may be used for sexual dysfunction, wherein when wishing to be aroused, a person uses remote-control unit 402 or palmtop 407, or another remote-control unit, for monitoring.

### Example 16

### Narcotic Rehabilitation

When using device 400 or 460, having status sensors, to determine and report the amount of specimen remaining in the specimen reservoir, for example, on display on remote-control unit 402 or palmtop 407, or another remote-control unit, the user may observe and actively participate in the specimen sampling rate.

### Example 17

Taking blood samples from people with mental disturbance (especially children) is a challenging task. Their unexpected physical behavior and lack of mental control can put on danger the use of injections. However, using a device such as shown in FIG. 16 can produce the same results at no risk.

### Example 18

### Controlled Specimen Sampling in the Treatment of Eating Disorders

Eating disorders are characterized by a persistent pattern of aberrant eating or dieting behavior. These patterns of eating behavior are associated with significant emotional, physical, and relational distress, as defined by the Academy Of Eating Disorder. Some common eating disorders are described below:

1. Anorexia Nervosa is defined as a serious, potentially life-threatening eating disorder characterized by self-starvation and excessive weight loss. Conventional, orally administered specimen treatment may include citalopram (Selective Serotonin Reuptake Inhibitor, 20 mg), fluoxetine hydrochloride (antidepressant, for relapse prevention during maintenance therapy, and olanzapine (atypical antipsychotic).

In accordance with the present invention, an oral device for controlled specimen sampling and a method for controlled specimen sampling, as taught in conjunction with any one of FIGS. 8A-14D may be applied in conjuction with the treatment.

2. Bulimia Nervosa is defined as a serious, potentially life-threatening eating disorder, characterized by a cycle of bingeing and compensatory behaviors such as self-induced vomiting designed to undo or compensate for the effects of binge eating. Conventional, orally administered drug treatment may include Fluoxetine (60 mg/day).

In accordance with the present invention, an oral device for controlled specimen sampling and a method for controlled specimen sampling, as taught in conjunction with any one of FIGS. 8A-14D may be applied.

The controlled sampling rate, which aims at maintaining a substantially constant specimen concentration in the blood by proper drug delivery, may be more effective at preventing binges and purges, then conventional, orally administered drug.

Furthermore, the ability to program the oral device for controlled specimen sampling for chronotherapeutic delivery may be applied, to program the device functioning according to the known times of binges, or of purges.

3. Obesity, or overweight are conventionally treated by L-tryptophan (essential amino acid, 1-3 g administered 1 h before a plated meal), Sibutramine (serotonin-noradrenaline reuptake inhibitor, 10-20 mg daily), and (or) bitter substances like nicotine.

In accordance with the present invention, an oral device for controlled specimen sampling and a method for controlled specimen sampling, as taught in conjunction with any one of FIGS. 8A-14D may be applied, with advantages similar to those cited in item (2).

4. Binge Eating Disorder and (or) Compulsive Overeating relate to any one of the following:

i. eating in a manner which is out of control;

ii. eating an unusually large amount of food;

iii. eating very quickly;

iv. eating to the point of feeling highly uncomfortable;

v. eating large amounts of food, even when not hungry;

vi. eating alone because of embarrassment due to the eating habits and quantities; and

vii. feeling disgusted, depressed, and guilty after overeating.

Binge eating disorder is conventionally treated with antidepressants, and zonisamide (antiepileptic drug, 100-600 mg/day).

In accordance with the present invention, an oral device for controlled specimen sampling and a method for controlled specimen sampling, as taught in conjunction with any one of FIGS. 8A-14D may be applied, with advantages similar to those cited in item (2).

5. Eating Disorders Not Otherwise Specified (EDNOS), which are defined as variants of anorexia nervosa or bulimia nervosa, as they do not meet the diagnostic criteria for anorexia nervosa or bulimia nervosa, but require treatment, nonetheless. Examples include women who would meet the criteria for anorexia nervosa, save for the fact that they continue to menstruate, individuals who regularly purge but do not binge eat, and individuals who nearly meet criteria for bulimia nervosa, but binge eat less than twice a week. EDNOS can be a serious, potentially life-threatening eating disorder. It is conventionally treated orally administered by ondansetron (serotonin receptor antagonist, 16 microg/kg twice daily), which is also effective for alcohol addiction, and (or) topiramate (anticonvulsant, 100 mg/day-range, 25-400 mg/day).

In accordance with the present invention, an oral device for controlled specimen sampling and a method for controlled specimen sampling, as taught in conjunction with any one of FIGS. 8A-14D may be applied, with advantages similar to those cited in item (2).

### Example 19

### Treatment to Quit Smoking

In accordance with the present invention, an oral device for controlled specimen sampling smoke sensing and a method for controlled specimen sampling and smoke sensing, as taught in conjunction with any one of FIGS. 8A-14D may be used for providing a controlled dose of nicotine or bupropion hydrochloride to assist a person trying to quit smoking. As a result of the intraoral delivery, a desired level of bupropion hydrochloride in the blood stream may be achieved and the noradrenergic and dopaminergic pathways in the brain are stimulated. In consequence, the sense of boredom and latency, which causes people to light a cigarette, is satiated by bupropion hydrochloride.

### Example 20

### Treatment of Alcoholism

Conventional treatment of alcohol may use, for example, orally administered ondansetron (serotonin receptor antagonist, 4 to 16 microg/kg twice daily). Certain serotonin receptors in the brain may have an effect on how alcohol impacts the brain. Ondansetron affects those receptors and has been shown to reduce alcohol consumption. In accordance with the present invention, an oral device for controlled specimen sampling and a method for controlled specimen sampling, as taught in conjunction with any one of FIGS. 8A-14D may be used for providing a controlled dose of ondansetron to assist a person trying to overcome a dependence on alcohol.

### Example 21

### Bad Breath Treatment

Anearobic bacteria on the surface of tongue and the throat cause bad breath, by breaking down proteins at a very high rate. The amino acids Cystein and Methionine, both rich in sulfur, are by-products of the process, and are delivery to the tongue and throat as Hydrogen Sulfide, methyl mercaptan, or other odorous substances (know as Volatile Sulfur Compounds VSC), producing bad breath. Conventional treatment relies on oral administration of any one of 0.2% chlorhexidine solution, chlorhexidin 0.05% plus etylpyridinium chloride 0.05% plus zinc lactate 0.14%, Zinc Gluconate, OXYD-8, or a mint flavoring. In accordance with the present invention, an oral device for controlled specimen sampling and a method for controlled specimen sampling, as taught in conjunction with any one of FIGS. 8A-14D may be applied. A controlled treatment regimen may include a controlled delivery of any one of 0.2% chlorhexidine solution, chlorhexidin 0.05% plus etylpyridinium chloride 0.05% plus zinc lactate 0.14%, Zinc Gluconate, OXYD-8, or a mint flavoring.

### Example 22

### Personalized Specimen Collection Based on DNA Analysis

Specimen sampling schedule may be based on DNA reconstruction and analysis, to match each patient's DNA. DNA parameters may be processed prior to the specimen collection, or during it, to define the best specimen collection policy for a particular patient. A-DNA dependent delivery schedule may occur, for example, in consequence to a determination that the patient's DNA includes a gene that makes that patient more susceptible to certain diseases, such as, breast cancer, or heart attacks.

### Example 23

### Personalized Specimen Collection Based on Physical Parameters and Personal History

Specimen sampling schedule may be based on physical-parameters and personal-history analyses, so as to be tuned to a specific patient. Physical-parameters and personal-history analyses may include patient's weight, height, age, gender, physiological history, medical status, other medication administrated simultaneously, blood pressure, blood analysis and the like. These parameters may be processed prior to the specimen collection, or during it, to define the specimen collection policy that will achieve best results for a particular patient.

### Example 24

### Permeation Enhancer

The buccal epithelium is similar in structure to other stratified epithelia of the body, and enhancers used to improve specimen permeation in other absorptive mucosae have been shown to work in improving buccal specimen penetration as well. (Shojaei, A. H., “Buccal Mucosa As A Route For Systemic Drug Delivery”: A Review, Faculty of Pharmacy and Pharmaceutical Sciences, University of Alberta, Edmonton, Alberta, Canada T6G 2N8). Some examples of permeation enhancer are: 23-lauryl ether, Aprotinin, Azone, Benzalkonium chloride, Cetylpyridinium chloride, Cetyltrimethylammonium bromide, Cyclodextrin, Dextran sulfate, Lauric acid, Lauric acid/Propylene glycol, Lysophosphatidylcholine, Menthol, Methoxysalicylate, Methyloleate, Oleic acid, Phosphatidylcholine, Polyoxyethylene, Polysorbate 80, Sodium EDTA, Sodium glycocholate, Sodium glycodeoxycholate, Sodium lauryl sulfate, Sodium salicylate, Sodium taurocholate, Sodium taurodeoxycholate, Sulfoxides. Various alkyl glycosides. It is expected that during the life of this patent many relevant oral devices and methods of controlled specimen sampling will be developed and the scope of these terms is intended to include all such new technologies a priori.

### Example 25

### Veterinary Use

A device similar to a device shown in FIG. 16, and adopted to the specific animal oral structure can be placed inside the animal oral cavity monitoring the animal health in a timely and fully controlled manner. The results are logged inside the device built-in memory and/or transmitted in real-time to the farmer's supervising center.

### Example 26

### Lactate Sensor

The device can be used by individuals under a physical stress great enough to endanger their health, such as sportsmen, firefighters, soldiers, divers, patients recovering from heart illness or surgery or critically ill patients. Those users will benefit from a device that monitors their metabolic status, since they may collapse when passing from aerobic to anaerobic metabolism. This stage is detected by the amount of lactate generated by the organism. Thus, an intra-oral sensor of lactate can be life-saving for such individuals, by emitting an alarm when they are about to pass from aerobic to anaerobic state. As a result of the alarm, the user should rest in order to return to the aerobic status.

### Example 27

### Behavioral Therapy to Treat Addictions

The device can be used to treat addictions to food (causing obesity), smoking, alcoholic beverages and oral illicit drugs. The addicted person will receive such a device to be worn in the mouth. The device will detect the presence of the unwanted substance in the mouth and transmit the information to a remote control and therapeutic center. This center will gather the incoming data and process them. Afterwards, those data can be used to educate the addicted device user to change his/her behavior. If the device is not worn, that may be an indication that the user wishes to conceal his substance intake from the control center. A specific data pattern will be transmitted to the center whenever the device is not worn. Thus, not usage of the device is also an important information that will be known by the control center, and will tech that the user is not complying with the rehabilitation regimen.

It will thus be appreciated that an aspect of the present invention relates to a device for specimen sampling, the device being adapted for insertion into an oral cavity of a subject and the device comprising:

a reservoir containing a specimen;

a specimen sampler, in communication with the reservoir, for providing the specimen sampling; and

a dental implement, configured for insertion into the oral cavity and for independently supporting the specimen reservoir, within the oral cavity.

Additionally, the dental implement may be selected from the group consisting of a prosthetic tooth crown, a dental bridge, a dental three-unit bridge, dental implant, partial dentures, full dentures, braces, a molar band, a night guard, and a mouth guard.

Moreover, the dental implement may be designed to be removably inserted to the oral cavity of a subject, for a period of between about 1 minute and about 1 day.

Alternatively, the dental implement may be designed to be removably inserted to the oral cavity of a subject, for a period of between about 1 day and about twenty years.

Additionally, the device may include an electronic component, selected from the group consisting of a specimen sampling mechanism, configured for providing the electronic, controlled specimen sampling, an electronic control unit, an electronic sensor, and an electronic analyzer.

Furthermore, the device may include at least two specimen reservoirs.

Moreover, each of the at least two specimen reservoirs may be controlled independently of the other.

Additionally, the device may include at least one local sensor, integrated with the device.

Furthermore, the at least one local sensor may be a physiological sensor, for specimen sampling responsive to measurements of the sensor.

Moreover, the local physiological sensor may be selected from the group consisting of a sensor for a concentration of the specimen, a sensor for glucose concentration, a sensor for a metabolite concentration, a sensor for an electrolyte concentration, a sensor for a pH level, a temperature sensor, a heartbeat sensor, a heart rate sensor, a lactate sensor, and a snoring sensor.

Additionally, the local physiological sensor may be a sensor for a fluid selected from the group consisting of gingival fluid, blood, interstitial fluid, saliva, sputum, and sweat.

Furthermore, the at least one local sensor may be a status sensor, for ensuring that the device is in proper operating condition.

Moreover, the local status sensor may be selected from the group consisting of a sensor for cumulated specimen in the specimen reservoir, a sensor for specimen flow rate, a sensor for power source condition, and a sensor for short-circuit detection.

Furthermore, the device may include at least one communication component, selected from the group consisting of a receiver, a transmitter, and a transceiver.

Moreover, the communication component may provide communication with a personal extracorporeal system, selected from the group consisting of a remote control unit, a computer system, a telephone, a mobile phone, a palmtop, a PDA, a laptop, and a combination thereof.

Additionally, the personal extracorporeal system may be adapted to provide communication between the device and a monitoring center.

Furthermore, the communication component may provide communication with at least one remote sensor.

Moreover, the local physiological sensor may be selected from the group consisting of a sensor for a concentration of the specimen, a sensor for glucose concentration, a sensor for a metabolite concentration, a sensor for an electrolyte concentration, a sensor for a pH level, an oxygen sensor, a temperature sensor, a heartbeat sensor, a heart rate sensor, a blood-pressure sensor, and a snoring sensor, a sensor for the presence of food, such as, carbohydrates, fat, juice, milk, and (or) dairy products, an alcohol sensor, and a tobacco sensor.

Additionally, the device may be adapted for a specimen collection selected from the group consisting of sublingual collection, labial mucosa collection, and soft-palatal collection.

It will thus furthermore be appreciated that another aspect of the present invention relates to a method for specimen sampling, comprising:

providing a device for specimen sampling, the device being adapted for insertion into an oral cavity of a subject and the device comprising:
a reservoir containing a specimen;
a specimen sampler, in communication with the reservoir, for providing the specimen sampling; and
a dental implement, configured for insertion into the oral cavity and for independently supporting the specimen reservoir, within the oral cavity;

mounting the reservoir on the dental implement; and

inserting the dental implement into the oral cavity.

As used herein the terms “generally” and “about” refer to ±30%.

As used herein the term “substantially” refers to ±10%.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

What is claimed is:

1. A device for specimen sampling, the device being adapted for insertion into an oral cavity of a subject and comprising:
a reservoir containing a specimen;
a specimen sampler, in communication with the reservoir, for providing the specimen sampling; and
a dental implement, configured for insertion into the oral cavity and for independently supporting the specimen reservoir, within the oral cavity.

2. The device of claim 1, wherein the dental implement is selected from the group consisting of a prosthetic tooth crown, a dental bridge, a dental three-unit bridge, dental implant, partial dentures, full dentures, braces, a molar band, a night guard, and a mouth guard.

3. The device of claim 1, wherein the dental implement is designed to be removably inserted to the oral cavity of a subject, for a period of between about 1 minute and about 1 day.

4. The device of claim 1, wherein the dental implement is designed to be removably inserted to the oral cavity of a subject, for a period of between about 1 day and about twenty years.

5. The device of claim 1, and further including an electronic component, selected from the group consisting of a specimen sampling mechanism, configured for providing the electronic, controlled specimen sampling, an electronic control unit, an electronic sensor, and an electronic analyzer.

6. The device of claim 5, and further including at least two specimen reservoirs.

7. The device of claim 6, wherein each of the at least two specimen reservoirs is controlled independently of the other.

8. The device of claim 1, and further including at least one local sensor, integrated with the device.

9. The device of claim 8, wherein the at least one local sensor is a physiological sensor, for specimen sampling responsive to measurements of the sensor.

10. The device of claim 9, wherein the local physiological sensor is selected from the group consisting of a sensor for a concentration of the specimen, a sensor for glucose concentration, a sensor for a metabolite concentration, a sensor for an electrolyte concentration, a sensor for a pH level, a temperature sensor, a heartbeat sensor, a heart rate sensor, a lactate sensor, and a snoring sensor.

11. The device of claim 9, wherein the local physiological sensor is a sensor for a fluid selected from the group consisting of gingival fluid, blood, interstitial fluid, saliva, sputum, and sweat.

12. The device of claim 8, wherein the at least one local sensor is a status sensor, for ensuring that the device is in proper operating condition.

13. The device of claim 12, wherein the local status sensor is selected from the group consisting of a sensor for cumulated specimen in the specimen reservoir, a sensor for specimen flow rate, a sensor for power source condition, and a sensor for short-circuit detection.

14. The device of claim 1, and further including at least one communication component, selected from the group consisting of a receiver, a transmitter, and a transceiver.

15. The device of claim 14, wherein the communication component provides communication with a personal extracorporeal system, selected from the group consisting of a remote control unit, a computer system, a telephone, a mobile phone, a palmtop, a PDA, a laptop, and a combination thereof.

16. The device of claim 15, wherein the personal extracorporeal system is adapted to provide communication between the device and a monitoring center.

17. The device of claim 14, wherein the communication component provides communication with at least one remote sensor.

18. The device of claim 9, wherein the local physiological sensor is selected from the group consisting of a sensor for a concentration of the specimen, a sensor for glucose concentration, a sensor for a metabolite concentration, a sensor for an electrolyte concentration, a sensor for a pH level, an oxygen sensor, a temperature sensor, a heartbeat sensor, a heart rate sensor, a blood-pressure sensor, and a snoring sensor, a sensor for the presence of food, an alcohol sensor, and a tobacco sensor.

19. The device of claim 1, adapted for a specimen collection selected from the group consisting of sublingual collection, labial mucosa collection, and soft-palatal collection.

20. A method for specimen sampling, comprising:
providing a device for specimen sampling, the device being adapted for insertion into an oral cavity of a subject and comprising:
a reservoir containing a specimen;
a specimen sampler, in communication with the reservoir, for providing the specimen sampling; and
a dental implement, configured for insertion into the oral cavity and for independently supporting the specimen reservoir, within the oral cavity;
mounting the reservoir on the dental implement; and
inserting the dental implement into the oral cavity.

21. The method of claim 20, wherein the dental implement is selected from the group consisting of a prosthetic tooth crown, a dental bridge, a dental three-unit bridge, dental implant, partial dentures, full dentures, braces, a molar band, a night guard, and a mouth guard.

22. The method of claim 20, wherein the dental implement is designed to be removably inserted to the oral cavity of a subject, for a period of between about 1 minute and about 1 day.

23. The method of claim 20, wherein the dental implement is designed to be removably inserted to the oral cavity of a subject, for a period of between about 1 day and about twenty years.

24. The method of claim 20, and further including an electronic component, selected from the group consisting of a specimen sampling mechanism, configured for providing the electronic, controlled specimen sampling, an electronic control unit, an electronic sensor, and an electronic analyzer.

25. The method of claim 24, and further including at least two specimen reservoirs.

26. The method of claim 25, wherein each of the at least two specimen reservoirs is controlled independently of the other.

27. The method of claim 20, and further including at least one local sensor, integrated with the device.

28. The method of claim 27, wherein the at least one local sensor is a physiological sensor, for specimen sampling responsive to measurements of the sensor.

29. The method of claim 28, wherein the local physiological sensor is selected from the group consisting of a sensor for a concentration of the specimen, a sensor for glucose concentration, a sensor for a metabolite concentration, a sensor for an electrolyte concentration, a sensor for a pH level, a temperature sensor, a heartbeat sensor, a heart rate sensor, a lactate sensor, and a snoring sensor.

30. The method of claim 28, wherein the local physiological sensor is a sensor for a fluid selected from the group consisting of gingival fluid, blood, interstitial fluid, saliva, sputum, and sweat.

31. The method of claim 27, wherein the at least one local sensor is a status sensor, for ensuring that the device is in proper operating condition.

32. The method of claim 31, wherein the local status sensor is selected from the group consisting of a sensor for cumulated specimen in the specimen reservoir, a sensor for specimen flow rate, a sensor for power source condition, and a sensor for short-circuit detection.

33. The method of claim 20, and further including at least one communication component, selected from the group consisting of a receiver, a transmitter, and a transceiver.

34. The method of claim 33, wherein the communication component provides communication with a personal extracorporeal system, selected from the group consisting of a remote control unit, a computer system, a telephone, a mobile phone, a palmtop, a PDA, a laptop, and a combination thereof.

35. The method of claim 34, wherein the personal extracorporeal system is adapted to provide communication between the device and a monitoring center.

36. The method of claim 33, wherein the communication component provides communication with at least one remote sensor.

37. The method of claim 28, wherein the local physiological sensor is selected from the group consisting of a sensor for a concentration of the specimen, a sensor for glucose concentration, a sensor for a metabolite concentration, a sensor for an electrolyte concentration, a sensor for a pH level, an oxygen sensor, a temperature sensor, a heartbeat sensor, a heart rate sensor, a blood-pressure sensor, and a snoring sensor, a sensor for the presence of food, an alcohol sensor, and a tobacco sensor.

38. The method of claim 20, adapted for a specimen collection selected from the group consisting of sublingual collection, labial mucosa collection, and soft-palatal collection.
